(19) United States
(12) Patent Application Publication
(10) Pub. No.: US 2022/0288227 A1
(43) Pub. Date: Sep. 15, 2022

(54) Ultrasound Responsive Microbubbles And Related Methods

(71) Applicant: The Trustees of the University of Pennsylvania, Philadelphia, PA (US)

(72) Inventors: Rajarshi Chattaraj, Philadelphia, PA (US); Daeyeon Lee, Wynnewood, PA (US); Chandra M Sehgal, Wayne, PA (US); Daniel A Hammer, Villanova, PA (US)

(21) Appl. No.: 17/695,612

(22) Filed: Mar. 15, 2022

Related U.S. Application Data
(60) Provisional application No. 63/161,056, filed on Mar. 15, 2021.

(51) Int. Cl. A61K 47/69; A61K 41/00; A61K 9/00; A61K 45/06

(52) U.S. Cl. CPC A61K 47/6925; A61K 41/0033; A61K 9/0009; A61K 45/06

(57) ABSTRACT

A microbubble composition, comprising: a plurality of microbubbles, a microbubble comprising a noble gas and/or perfluorocarbon encapsulated within a shell that comprises one or more of a lipid, a protein, or a polymer, and a microbubble optionally defining a cross-sectional dimension in the range of from about 0.5 to about 20 micrometers. A method, comprising forming a composition according to the present disclosure. A method, comprising administering a microbubble composition according to the present disclosure to a subject, the composition optionally comprising echogenic phospholipid microbubbles. A method, comprising: (a) identifying, with the application of energy, the location of a microbubble composition according to the present disclosure, the energy optionally being ultrasound, (b) controllably effecting rupture of microbubbles of a microbubble composition of the present disclosure, the rupture optionally being effected by application of ultrasound, or both (a) and (b).

### RELATED APPLICATIONS

This application claims priority to and the benefit of U.S. patent application No. 63/161,056, “Ultrasound Responsive Noble Gas Microbubbles” (filed Mar. 15, 2021), the entirety of which application is incorporated herein for any and all purposes.

### GOVERNMENT RIGHTS

This invention was made with government support under EB022612 and OD016310 awarded by the National Institutes of Health. The government has certain rights in the invention.

### TECHNICAL FIELD

The present field relates to the field of microbubbles and to the field of encapsulated noble gases.

### BACKGROUND

Noble gases have been shown to have antiapoptotic effects in the treatment of hypoxia ischemia related injuries. Existing in vivo gas delivery, however, is performed in a systemic manner (e.g., via inhalation, which leads to reduced efficacy at the injury site. Existing methods of delivering encapsulated noble gases also suffer from low payload and little diagnostic signal. Accordingly, there is a long-felt need in the art for improved compositions of encapsulated noble gases and for related methods of making and using such compositions.

### SUMMARY

In meeting the described long-felt needs, the present disclosure first provides a microbubble composition, comprising: a plurality of microbubbles, a microbubble comprising a noble gas and/or a perfluorocarbon encapsulated within a shell that comprises one or more of a lipid, a protein, or a polymer, and a microbubble optionally defining a cross-sectional dimension in the range of from about 0.5 to about 20 micrometers.

Also provided is a method, comprising: forming a composition according to the present disclosure, e.g., according to any one of Embodiments 1-15.

Further provided is a method, comprising administering a microbubble composition according to the present disclosure (e.g., according to any one of Embodiments 1-15) to a subject, the composition optionally comprising echogenic phospholipid microbubbles.

A method, comprising: (a) identifying, with the application of energy, the location of a microbubble composition according to the present disclosure (e.g., any one of Embodiments 1-15), the energy optionally being ultrasound, (b) controllably effecting rupture of microbubbles of a microbubble composition of the present disclosure (e.g., any one of Embodiments 1-15), the rupture optionally being effected by application of ultrasound, or both (a) and (b).

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1A-1B. Structures of DSPC and DBPC (1A), and DSPE-PEG (1B), where n is 45 for PEG2000 and 113 for PEG5000.

FIGS. 2A-2B. Particle size distributions after 1-2 h (purple) and 5 d (red) (2A), and representative micrographs (AB) of noble gas MBs formulated by shaking, with DSPC+DSPE-PEG5000 as shell material. Scale bar: 20 μm.

FIGS. 3A-3B. Particle size distributions after 1-2 h (purple) and 5 d (red) (3A), and representative micrographs (3B) of noble gas MBs formulated by sonication/centrifugation, with DBPC:DSPE-PEG5000 (9:1) as shell material. Scale bar: 20 μM.

FIG. 4. Fluorescence signal of 90 μL of 30 μM cryptophane (TTPC) without (black) and with 50× diluted (red) and 10× diluted (blue) as-prepared XeMBs to a total volume of 100 μL. Inset: Structure of TTPC.

FIG. 5. Decay of non-linear contrast signal intensity from noble gas microbubbles over time under continuous exposure to pulsed ultrasound at 3.4 MHz and a mechanical index of 0.12, in PBS (black) and 50% FBS (blue). Inset: Representative still frames at 0 min (left) and 5 min (right), for MBs in PBS (top) or 50% FBS (bottom).

FIGS. 6A-6B: Particle size distributions after 1-2 h (purple) and after 5 d (red) and representative micrographs of the same (inset) of a scaled-up sonication/centrifugation formulation sample of DBPC:DSPE-PEG5000 (9:1) XeMBs. Scale bar: 20 μm.

FIG. 7. In vivo signal from retro-orbital injection of 0.03 mL XeMBs from the left ventricle of the heart (HI-H3) and kidney (K1-K3) of a mouse. 1 and 2 show representative still frames from before (1) and a few seconds after (2) injection respectively. H3 and K3 show the quantified signal with time, where 0 s indicates the time at which the contrast agent arrives at the region of interest. Imaging is carried out at 18 MHz and 10% power. The double peak seen in H3 is due to flushing of the agent before infusion of saline.

FIG. 8 provides a schematic of microbubble/phantom flow imaging setup.

FIG. 9 provides an exemplary size distribution of MBs with PFB and Xe cores and 9:1 phospholipid:DSPE-PEG2000 (top) and representative micrographs of PFB and Xe MBs (bottom).

FIG. 10 provides (top) representative ultrasound images of PFB MBs with different 90 mol % primary phospholipids in the shell and (bottom) ultrasound contrast gradient along the length of flow of DPPC-, DSPC- and DBPC-stabilized PFB MBs.

FIG. 11 provides mean ultrasound contrast gradient along length of flow of DPPC-, DSPC- or DBPC-stabilized PFB MBs at different flow rates as a function of changing mechanical index from lowest (0.12) to highest (0.8).

FIG. 12 provides mean ultrasound contrast gradient along the length of flow of DPPC-, DSPC- or DBPC-stabilized PFB MBs at different MIs as a function of changing flow rate from lowest (F1) to highest (F6).

FIG. 13 provides (top) representative images of Xe MBs with 9:1 DBPC:DSPE-PEG2000 in the shell imaged in non-linear contrast mode at the lowest (FLow) and highest (FHigh) flow rates using the lowest (0.12) and highest (0.8) mechanical indexes studied and (bottom) mean ultrasound contrast gradient along length of flow of DBPC-stabilized Xe MBs, (left) at different mechanical indices as a function of changing flow rate from lowest (F1) to highest (F6). MI: 0.12 (blue), 0.18 (green), 0.31 (red), 0.5 (gray) and 0.8 (black), and (right) at different flow rates as a function of changing acoustic contrast of fluorocarbon and xenon MBs under flow.

FIG. 14 provides (left) representative ultrasound images from static imaging in a pipet bulb immersed in water of different MBs at a concentration of ˜2.5×10⁵/mL (PFB) or 1×10⁶/mL (Xe) in phosphate-buffered saline and (right) representative ultrasound images from static imaging in a latex tube embedded in silicone, of different MBs at concentrations of 1×10⁶/mL (PFB) or ˜3×10⁶/mL (Xe). Imaging was carried out at 3.4 MHz and MI=0.12. Images were taken after 1-2 s of ultrasound exposure. DBPC=1,2-dibehenoyl-sn-glycero-3-phosphocholine; DPPC=1,2-dipalmitoyl-sn-glycero-3-phosphocholine; DSPC=1,2-distearoyl-sn-glycero-3-phospho choline (MB=microbubble; MI=mechanical index; and PFB=perfluroroburane).

FIGS. 15A-15C illustrates neuroprotective efficacy of xenon microbubbles in the porcine model of traumatic brain injury. There is marked decrease in edema from Day 1 magnetic resonance imaging (MRI) (15A) to Day 5 MRI (15B) in the xenon-treated group. On the right, graph is showing percentage change in volumes (cm³) from Day 1 MRI to Day 5 MRI of edema, core, and total volume in the perfluorobutane (PFB)-treated (gray) and xenon-treated groups (n=3 each). The statistical significance of the difference in the percentage change between PFB-treated versus xenon-treated groups is assigned as * for p≤0.03, ** for p≤0.01, and ns (not significant).

FIGS. 16A-16C illustrates histology in xenon microbubbles treated group versus perfluorobutane (PFB) microbubble treated group. Vessels (black arrows) adjacent to the site of injury (asterisks) in the xenon condition showed less endothelial proliferation and perivascular inflammation (including neutrophils, lymphocytes, and macrophages) compared with the PFB-treated group. Hematoxylin and eosin stain. Scale bar applies to all images. On the right, graphs are showing the difference in endothelial proliferation score (16A) and perivascular inflammation score (16B) between the PFB-treated (n=2) and xenon-treated groups (n=3). The statistical significance of the difference in scores between control versus xenon-treated groups is assigned as * for p≤0.02 and ns (not significant).

FIGS. 17A-17C. 17A. Particle size distributions of ArMBs with a shell composition of DBPC:DSPE-PEG5000 (9:1). Red curve represents a starting stock of 1 mL of 10 mg/mL lipid solution while the purple curve represents scale up with 10× the starting volume. Inset shows a representative micrograph of scaled-up ArMBs diluted 20× before imaging. 17B. In vivo signal from retro-orbital injection of 0.03 mL XeMBs from the kidney of a mouse, showing representative still frames from before and a few seconds after injection. 17C. Quantified signal with time, where 0 s indicates the time at which the contrast agent is injected. Imaging is carried out at 18 MHz and 10% power. Inset: Zoomed in section of the decay curve showing where the contrast peaks. Contrast signal from XeMBs and ArMBs are comparable.

### DETAILED DESCRIPTION OF ILLUSTRATIVE EMBODIMENTS

The present disclosure may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable, and it should be understood that steps may be performed in any order.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. All documents cited herein are incorporated herein in their entireties for any and all purposes.

Further, reference to values stated in ranges include each and every value within that range. In addition, the term “comprising” should be understood as having its standard, open-ended meaning, but also as encompassing “consisting” as well. For example, a device that comprises Part A and Part B may include parts in addition to Part A and Part B, but may also be formed only from Part A and Part B.

Noble gases, especially xenon (Xe), have been shown to have antiapoptotic effects in the treatment of hypoxia ischemia related injuries. Currently, in vivo gas delivery is systemic and performed through inhalation, which leads to reduced efficacy at the injury site. Localized delivery by encapsulation of Xe within the bilayer of liposomes suffers from low payload and little diagnostic signal. In this report, we provide a first demonstration of the encapsulation of pure Xe, Ar, or He in phospholipid-coated sub-10 μm microbubbles. We determine the optimal shell compositions and preparation techniques for formulating stable, echogenic microbubble formulations. We find that DSPC with DSPE-PEG5000 can produce stable microbubbles upon shaking, while DBPC blended with either DSPE-PEG2000 or DSPE-PEG5000 produces a high yield of microbubbles via a sonication/centrifugation method. Xe and Ar concentrations released from the microbubble suspension into the headspace of the storage vial are measured using GC-MS, while Xe release directly in solution is detected by the fluorescence quenching of a Xe-sensitive cryptophane molecule. These microbubbles provide excellent contrast in vitro for several minutes under physiological conditions. In addition, bubble production is found to be amenable to scale-up while maintaining their size distribution and stability. Finally, we show a first instance of administration of a bolus of pure Xe microbubbles providing significant ultrasound contrast in a mouse in a pre- and post-lung setting (heart and kidney, respectively). Modulating shell composition thus provides evidence of the stable formulation of echogenic microbubbles with not just Xe, but Ar and He as well, while also ensuring sufficient in vivo circulation persistence of Xe microbubbles injected intravenously for theranostic applications.

In recent years, the therapeutic properties of noble gases have become a growing area of study. Combination therapies using xenon (Xe), in particular, provide ample evidence for cardioprotective and neuroprotective effects in hypoxic-ischemic injuries.

While the ability to freely diffuse through the blood-brain barrier without adverse effects make Xe an excellent anesthetic, the mechanism of the antiapoptotic action of Xe is still not completely understood. A proposed mechanism behind the neuroprotective effect of Xe relies on its blockage of N-methyl-D-aspartate (NMDA) receptors, thus preventing the excess build-up of Ca′ ions in the extracellular neural tissue, which leads to excitotoxicity. Because of its chemical inertness and high electron density, Xe is postulated to undergo spontaneous polarization to bind to the active sites of enzymes and receptors, rendering them active or inactive. Xe is the most potent of all noble gases because it is the most polarizable noble gas.

Other noble gases have shown positive effects in various contexts. For example, controlled doses of argon (Ar) have been demonstrated to reduce neuronal death in vitro in models of an oxygen-glucose deprivation (OGD) and traumatic brain injury (TBI), and post-cardiac arrest induced neuronal damage in vivo.

Similarly, helium (He) has been effective against TBI and in vivo models of acute ischemic injury, although some negative impact of He such as OGD injury in renal cells and intra-tumoral growth have been reported. Many of these studies involving noble gases offer intriguing potential for their use in medical gas therapy for stroke, cardiac arrest, or even cancer.

In most of these studies, noble gases that are used as therapeutic agents are delivered to the subject through inhalation. Indeed, clinical trials regarding Xe inhalation to treat brain injuries have been ongoing. The primary problem of this method is that it relies on systemic delivery of the gases, which may drastically reduce their local therapeutic efficiency. Moreover, Xe, while being the most effective noble gas, has the disadvantage of being expensive and inadequately available for continuous use.

To address these issues, Huang and coworkers trapped Xe within the hydrophobic core of liposome bilayers to deliver the gas locally to the injury site in models of stroke. These liposomes were effective in reducing neuronal cell apoptosis when they were injected directly into the carotid artery or through the tail vein, and gas was released from the shell via ultrasound exposure at the carotid. Although this is one proof-of-concept to verify the therapeutic potential of Xe, making a carotid insertion may pose a challenge for treatment of acute stroke or similar ischemic conditions, with potential complications for patients. In addition, neither path of administration was reported to show in vivo ultrasound signal at the delivery site, limiting their utility as theranostic agents. The ultimate goal is to locally deliver high quantities of gases via intravenous injection, while being able to image the delivery vehicle through ultrasound at the location of the injury. In this sense, liposomes may not be the ideal agent due to their limited encapsulation efficiency and low in vivo ultrasound contrast. As an alternative, we propose microbubbles as a solution to these issues, since bubbles have almost their entire volume occupied by the gas, theoretically providing a ten- to hundred-fold increase in gas payload, in addition to being highly echogenic under ultrasound.

Microbubbles (MBs) are 1-10 μm particles with an internal gas core, stabilized by a lipid, polymer, or protein shell. The core can be a noble gas and/or a perfluorocarbon (PFC) gas, while the shell comprises a phospholipid and/or lipopolymer that reduces the surface tension at the aqueous/gas interface. MBs are primarily used for contrast enhanced ultrasound imaging due to their excellent scattering and non-linear volumetric oscillation properties on ultrasound application at clinical frequencies.

MBs can also be functionalized with targeting groups such as antibodies, peptides, and aptamers, thus having the potential for precise, image-guided drug or gene delivery. Of recent, MBs have been used as carriers for therapeutic gases like oxygen and nitric oxide.

Oxygen MBs, especially, have been successfully employed to treat a wide range of hypoxia-mediated injury models. As far as noble gases are concerned, a study reports the formulation and characterization of MBs with a Xe or a Xe/PFC mixed core.

Pure Xe MBs, however, were too large and non-echogenic in vivo. Since noble gases are significantly more water-soluble and/or smaller than perfluorocarbon molecules, the stable entrapment of Xe, Ar, and He is an added challenge, similar to that of oxygen, which is why a small percentage of PFC is often used as a stabilizing agent.

Herein, we report the design and functionality of pure noble gas microbubbles (NGMBs) as a function of phospholipid shell compositions and preparation methods of high frequency shaking and sonication/centrifugation. In addition to Xe, also explored are Ar and He MBs as well, while testing their echogenic properties in vitro. Finally, provided is testing of the in vivo imaging properties of a scaled-up and optimized formulation of pure Xe MBs.

Exemplary Results

Microbubble Formulation and Stability

The present disclosure provides, inter alia, MBs with high yields and stability over long periods of time, with the vast majority between 1-10 μm. As opposed to PFCs—the standard gas for contrast MBs—noble gases are either more water-soluble (Xe, Ar) or significantly smaller/permeable (Ar, He), making them more susceptible to dissolution into the surrounding aqueous medium. This characteristic can be supported y a shell of the MB that is sufficiently rigid and impermeable to maintain the gas in the core.

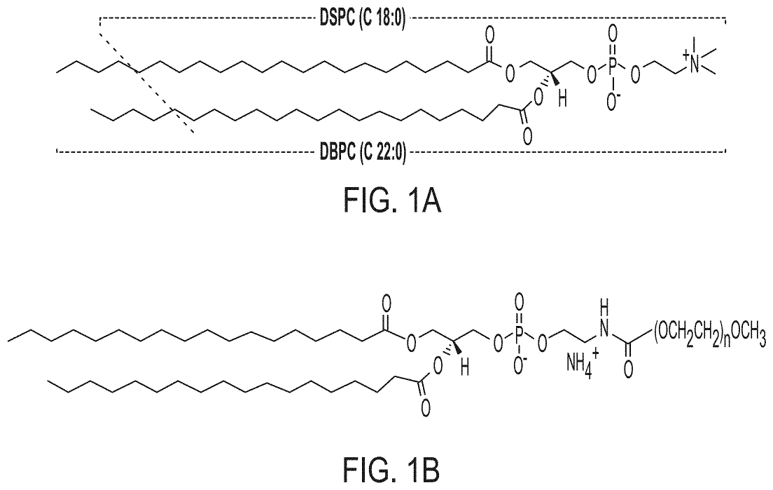

Within this framework, one can also consider the lipophilicity of the gas molecules; for instance, Xe is known to interact with hydrophobic tails of phospholipids, and subsequently fluidize the membrane. While DPPC (C16:0) (1,2-dipalmitoyl-sn-glycero-3-phosphocholine) is the most common primary phospholipid used for formulating PFC MBs, it has been shown to be less effective in encapsulating other relatively more soluble gases like oxygen; DSPC (C18:0) (1,2-distearoyl-sn-glycero-3-phosphocholine), with two extra carbon atoms in each acyl tail, has been found to be more robust. Furthermore, DBPC (C22:0) (1,2-dibehenoyl-sn-glycero-3-phosphocholine), with 22 C atoms, is known to form a rigid, coherent, gel phase below its melting temperature (74° C.). DSPC and DBPC are thus chosen as the primary phospholipids for the NGMB shell (FIG. 1).

A lipopolymer can be incorporated to enhance the steric stability and lower the immunogenic response of the particle. Polyethylene glycol (PEG) attached to a saturated phospholipid is a common example; DSPE-PEG2000 (1,2-distearoyl-sn-glycero-3-phosphoethanolamine-N4amino(polyethyleneglycol)-20001 (ammonium salt)) and DSPE-PEG5000 (1,2-distearoyl-sn-glycero-3-phosphoethanolamine-N-[methoxy(polyethylene glycol)-5000] (ammonium salt)) are therefore suitable.

The method of preparation also plays a role in determining the bubble yield and stability. High energy methods such as sonication can produce polydisperse yields, followed by lower-energy methods such as shaking/agitation, whereas microfluidic techniques produce a uniform size distribution, but with a significantly lower concentration while taking a longer time.

When MBs are used for imaging, size uniformity is highly important to obtain unique non-linear signals at specific frequencies of insonation. However, when MBs are intended for delivering their core gas, a broad size distribution does not significantly affect their performance. MBs can be greater than 1 μm in diameter so as to encapsulate a sufficient gas dose, but should be smaller than 12-15 μm so as to be able to flow through the small blood vessels which make up most of the circulatory network. At the same time, a distribution of 1-10 μm will also provide the maximum echogenicity due to resonance at clinical frequencies, which are usually between 1-10 MHz.

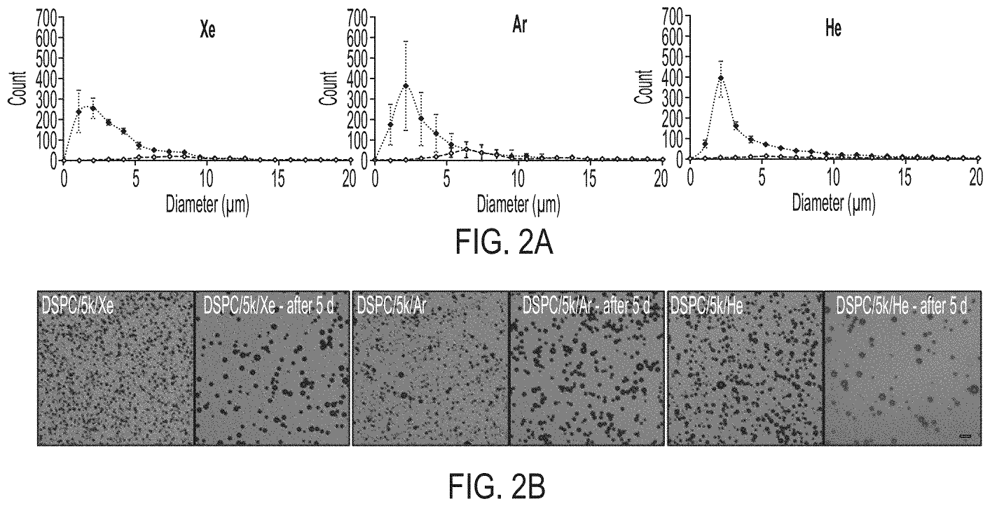

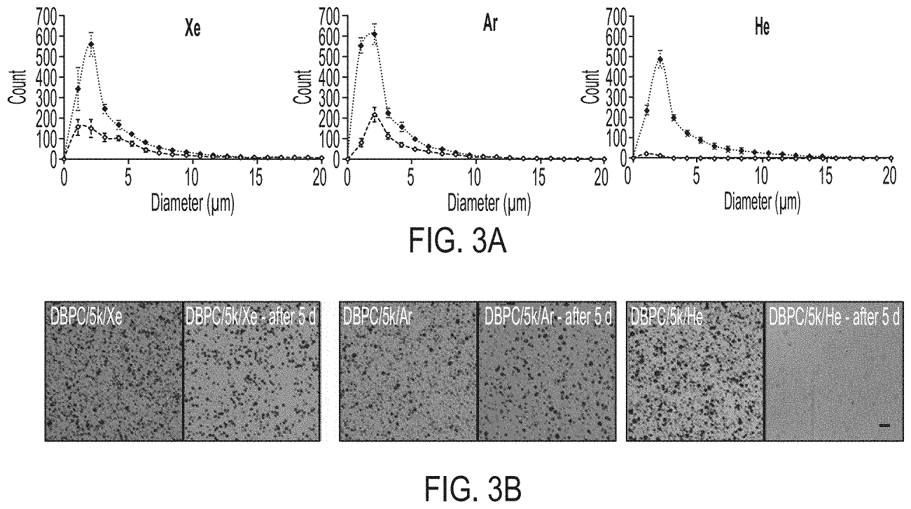

FIGS. 2 and 3 show optical micrographs of NGMBs and their size distributions formed by shaking and sonication/centrifugation using two different primary phospholipids and two different lipopolymers.

Shaking involves utilizing a widely used Vialmix® shaker for preparing commercially available Definity® bubbles, wherein gas and lipid solution are agitated in a sealed 2 mL vial for 45 s at ˜4300 Hz.

Sonication involves applying acoustic energy near the tip of a probe at the interface between gas and liquid, as employed by many prior studies. Sonicated bubbles are then centrifuged and washed to separate out the excess lipid solution at the bottom of the tube and the unstable foam at the top. One can compare shaking and sonication by first saturating the lipid solution with the gas, and then emulsifying immediately. The findings show that DSPC forms MBs with the most desired size/stability parameters while using the shaking protocol, while DBPC is more effective when using sonication/centrifugation.

DBPC MBs were a stable, high-yield MB combination. Without being bound to any particular theory, the cause for this may be linked to both the membrane residence time and packing efficiency of the shell material of the microbubble shell. The residence time depends heavily on the critical micelle concentration of the lipid: from DSPC to DBPC, as CMC decreases, the residence time increases by a factor of 4-10 per —CH₂ pair added to the acyl chain. Because DBPC has a higher membrane residence time than DSPC, the monolayer packing structure retains its integrity better for DBPC MBs. In fact, from C16:0 to C22:0, the inter-lipid cohesive energy is postulated to increase by approximately 50%. Additionally, sonication, which is the higher energy method, is likely more effective in overcoming the surface energy barrier to create a larger surface area, resulting in a greater yield of smaller bubbles. The lack of stability of smaller DSPC bubbles formed by sonication may be due to the open-to-atmosphere sonication setup, which can be rectified during scale-up.

While this overall trend applies to any MB with any gas (including perfluorocarbons), the comparative stabilities and size distributions are dependent on gas molecule size, water-solubility, and lipophilicity. As an example, DBPC appears capable of self-assembling around smaller droplets via shaking for Ar and He, but forms only comparatively large bubbles with Xe, possibly due to lower water-solubility of Ar and He dominating the surface energy barrier for a longer-chain lipid in this case.

These findings thus serve as a guide for formulating NGMBs with desired properties. There are two important aspects of these results. First, while calculating size distribution from images, we have not included particles below 0.8 μm in diameter, so as to show the distribution of micron-sized bubbles and not include nanobubbles and liposomes that are also likely to be present in the solution. Second, these results are based on a limiting case, in that all samples are prepared from a 1-1.3 mL initial volume of lipid solution, thus providing a framework for choosing lipids. For practical therapeutic use, production will have to be scaled up, and MB formulations could very well be more stable when stored in a 10-100× concentrated volume simply by virtue of there being more bubbles to reduce gas and lipid transfer gradients between the bubble and the aqueous phase. Hence, the reduction in MB concentration after 5 days, as seen in FIGS. 2 and 3, could be avoided by concentrating bubbles, as demonstrated later.

One may notice that changing the length of the PEG stabilizer affects the aggregation of microbubbles. MBs with DSPE-PEG2000 produce aggregates in some cases, whereas DSPE-PEG5000 keeps bubbles separated. While this is understandable in terms of greater steric repulsion due to longer PEG chain length, it is also slightly surprising, since DSPE-PEG2000 is widely used for contrast MB formulation with perfluorocarbons, and aggregation of such MBs have not been widely reported. Even though HeMBs with DSPE-PEG5000 are not stable after 5 days, we choose this composition for the sake of consistency with Xe and Ar formulations. Based on all of the above results, a shell composition of DBPC:DSPE-PEG5000 (9:1) for MBs made by sonication/centrifugation is used for all further experiments (Table 1, below).

| | Mean Diameter (μm) | | Concentration (#/mL) | |
|---|---|---|---|---|
| Gas | 1-2 h | 5 d | 1-2 h | 5 d |
| He | 4.64 ± 0.32 | — | 3.6 ± 0.18 × 10⁸ | — |
| Ar | 3.76 ± 0.6 | 4.54 ± 0.22 | 4.62 ± 0.19 × 10⁸ | 1.65 ± 0.16 × 10⁸ |
| Xe | 4.2 ± 0.16 | 4.83 ± 0.44 | 4.25 ± 0.45 × 10⁸ | 1.85 ± 0.2 × 10⁸ |

Table 1. Parameters for noble gas MBs formulated by sonication/centrifugation, with DBPC:DSPE-PEG5000 (9:1) shell material, and used for gas quantification and ultrasound experiments.

Noble Gas Quantification

Cryptophane Fluorescence Quenching

The next step after preparing stable NGMBs within the desirable size range is to both verify and quantify the type and amount of gas that is encapsulated. Common methods like gas chromatography-mass spectrometry (GC-MS) provide quantification of the gas released from the liquid into the headspace.

However, to directly detect the presence of gas released by MBs in solution, we use a class of compounds called cryptophanes which have inherent affinity towards Xe. Cryptophanes can encapsulate small molecules such as Xe, which leads to quenching of their native fluorescence.

To detect Xe, XeMBs are resuspended in PBS (without any infused Xe), and mixed with a water soluble cryptophane (tris(triazole propionic acid) cryptophane-A derivative, TTPC).

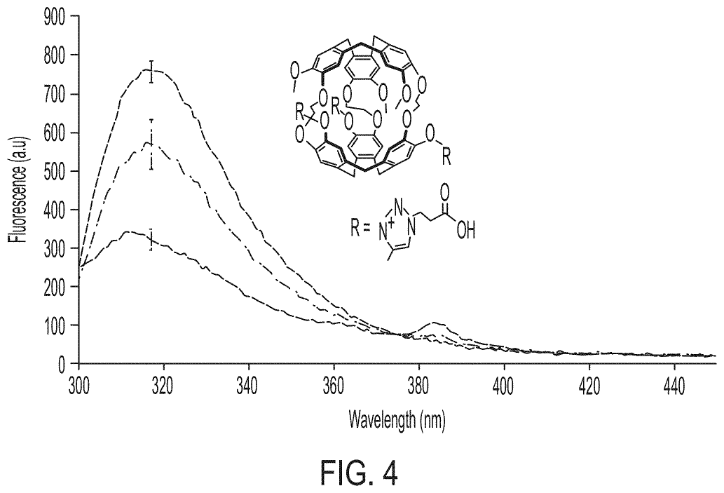

FIG. 4 shows the reduction of fluorescence as the concentration of bubbles is increased. This provides evidence that XeMBs can release their payload by passive dissolution when put in a Xe-desaturated media. However, at high Xe concentrations, as is the case with MBs, the dynamic range of fluorescence response is small and highly non-linear. Assuming a linear relationship between the concentration and fluorescence signal, the method gives ˜440 μL/10⁸ MBs based on a 10× dilution and ˜100 μL/10⁸ MBs based on a 50× dilution of MB solution. This method is thus a qualitative but direct way to detect dissolved Xe.

GC-MS

To quantify both Xe and Ar content in MBs, we use the more common way of indirect measurement of gas in the headspace of MB container, ideally after all the gas from the MBs has been released.

Gas chromatography/mass spectroscopy (GC-MS) is an effective way of characterizing the headspace contents. Previous reports have used this technique to quantify the amount of Xe in liposomes and MBs. Because bath-sonication is used as a common method for degassing solutions, Xe and Ar MBs are taken in sealed vials, and bath-sonicated to 1) break up the MBs, and 2) release the gas into the headspace. Gas from the headspace analyzed by GC-MS shows Xe and Ar peaks at similar retention times.

Based on calibration experiments performed for controlled volumes of pure gas, the amount of Xe present in a MB solution at a concentration of ˜10⁸ microbubbles/mL was determined to be 126±57 μL for XeMBs and 232±44 μL for ArMBs. It should be noted that a simple geometrical volume calculation of the total volume of 10⁸ MBs with an average diameter of 5 μm is approximately 26 μL. The excess amount of gas obtained is likely held mostly by sub-micron bubbles which are not taken into consideration when calculating the concentration of MBs (as mentioned in the previous section). This method of quantification has limitations in that it ignores the possibility of not all the gas being released into the headspace, as well as potential operator error in withdrawing small volumes of gas from the vial. Finally, the GC-MS setup does not allow detection of gases with molecular weights below 10—hence, it was not possible to quantify the amount of helium present in He MBs.

Ultrasound Imaging

After optimizing the size and stability parameters for NGMBs and established the type and quantity of their gas content, we next analyze their echogenicity under ultrasound. Since one may aim to use these MBs for clinical applications, a clinical imager (Zonare) is chosen to study their acoustic response.

A bubble concentration of ˜10⁷ per mL is first incubated in either PBS or 50% FBS (the latter, to observe if serum proteins have any effects on MB acoustic behavior) at 37° C. for 1 min to allow for temperature and protein interaction equilibration. Imaging is carried out at a low MI of 0.12 to minimize MB destruction, while being in a clinically relevant range.

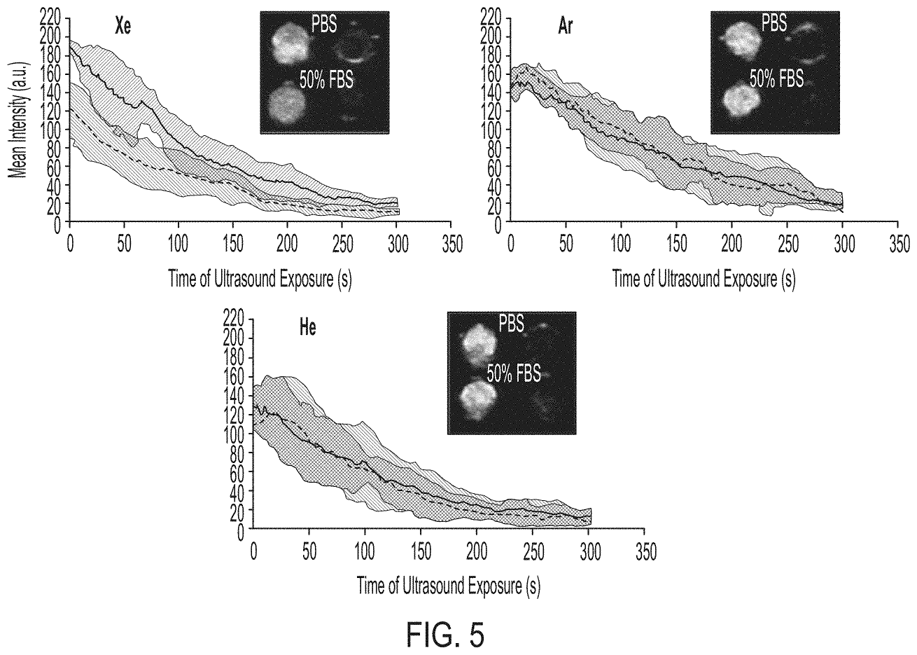

FIG. 5 shows that the all NGMBs are highly echogenic, and that significant contrast lasts for at least 3 min under continuous exposure to pulsed ultrasound in a static condition. This is a promising display of NGMBs as therapeutic agents that can be simultaneously imaged while the gas is being released. For ArMBs and HeMBs, serum proteins do not seem to affect the echogenicity appreciably. Interestingly, for XeMBs, the initial contrast is somewhat lower in 50% FBS than in PBS. More studies need to be carried out to affirm and explain this result, but one possible reason could be the predicted affinity of Xe for serum proteins.

Another explanation could stem from the ability of Xe to fluidize lipid membranes, which reports have indicated may allow lipids to rearrange, thus disrupting the packing structure and lowering the adsorption free energy for serum proteins, allowing for increased membrane mass to marginally damp the MB oscillation.

XeMB Formulation Scale-Up and In Vivo Ultrasound Imaging

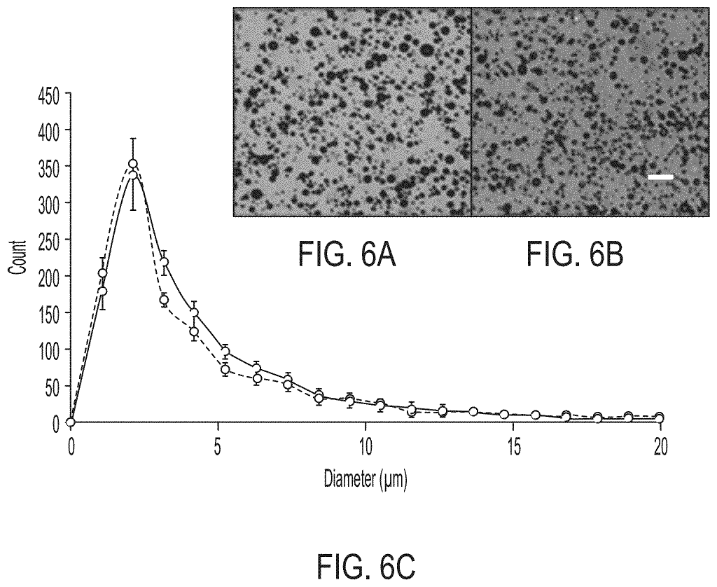

Next, we evaluate the potential of XeMBs in enhancing ultrasound images in a realistic physiological setting of blood flow including transit of the agent through the lungs. We choose XeMBs, since Xe is the gas currently most being explored. Additionally, since our system is based on a bolus injection (as opposed to continuous infusion, which will be the way for treating injuries), the concentration of MBs is scaled up. A larger volume of lipid solution (10 mL) is probe-sonicated and a larger number of MBs thus generated are concentrated using centrifugation to a small volume (˜4×10⁹/mL). FIG. 6 shows that these XeMBs have the desired size distribution, and are in fact, more concentration-stable than those made from a 1 mL initial lipid stock, as was mentioned before.

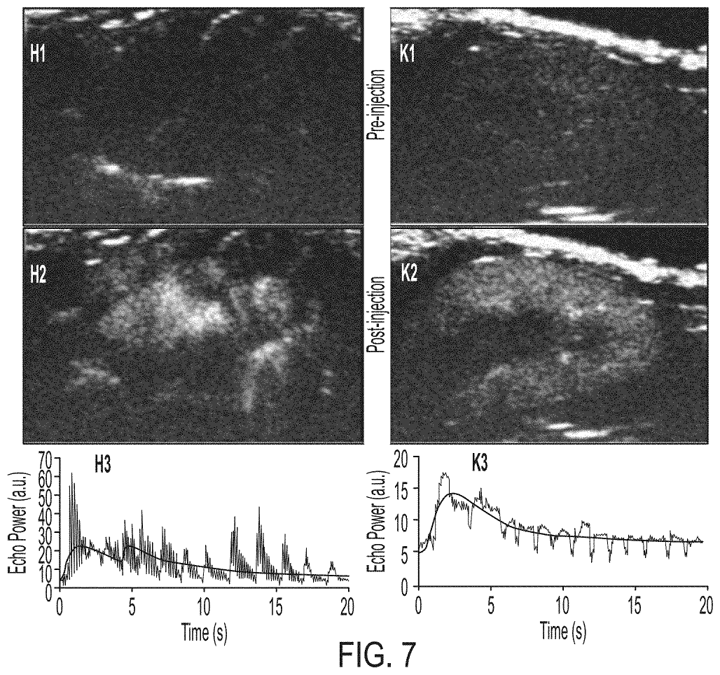

Freshly prepared XeMBs are injected into a mouse retro-orbitally, and the heart is imaged under high frequency ultrasound (18 MHz, Visualsonics) at 10% power. FIG. 7 shows XeMBs distinctly enhancing the cardiovascular contrast within seconds, subsequently retaining some reduced signal under ultrasound for at least 30 s. The contrast fades faster in the kidney, probably due to lower volume of the agent reaching the organ along with smaller vessels. This result demonstrates that XeMBs can perform local, image-guided gas delivery using a venous injection.

Observations

The salient points of this work involve the feasibility of formulation of stable noble gas MBs and their potential as theranostic agents. Of the different noble gases, the use of Xe has been clinically explored—yet only a few studies so far report the localized delivery of Xe, primarily through liposomes. However, assuming 5 μm and 0.8 μm diameters for MBs and liposomes respectively, with a 6 nm liposome bilayer thickness, and adjusting for volume/number concentration ratio due to different MB and liposome size, MBs can encapsulate ˜50 times more gas than liposomes.

For delivery of gases, MBs have inherent advantage over liposomes, as stated before. As with early studies with another relatively water-soluble gas, oxygen, perfluorocarbons were employed to sufficiently solubilize the Xe to reduce its diffusion out of the bubble. Attempts to formulate MBs with pure Xe in that study seems to have yielded a larger size distribution and lack of in vivo echogenicity. Doping a noble gas with a PFC may pose its own challenges since the finite solubility of the gas in the PFC will limit the amount of NG released.

This disclosure demonstrates a straightforward way to stabilize pure Xe simply by using a higher chain length phospholipid in the shell, which we hypothesize will trap the Xe in the MBs long enough to reach the delivery site. Enhanced stability and retention may have to do with the rigid packing structure of the DBPC shell rather than the lipophilicity of Xe, as evidenced by the ability of the same lipid to stabilize other noble gases.

Furthermore, the effect of Xe in fluidizing the lipid membrane is probably limited when it comes to long-chain lipids. This is of note because MBs have to survive the oxygen gradient when passing through the lungs to reach their target. The fact that post-lung MBs are considerably echogenic in the kidney, and that the time required for signal to appear is marginally longer in the kidney than the heart (1-2 s), would indicate that most of the original structure is retained. Further study is necessary to determine the interplay of various parameters in the stability and extent of passive gas efflux from the bubble. Although previous theoretical models have emphasized the large chemical potential gradient for soluble gases such as oxygen to diffuse out of the bubble, there is no study on such aspects for “amphiphilic” gases such as Xe Such a model is all the more important to predict the fate of MB interaction with the bloodstream and the lungs, when injected intravenously, as demonstrated here, (instead of directly into a carotid line), for practical utility in therapeutic use.

Also examined were microbubble preparation methods. Unlike imaging, where the quantity of MBs required is low, delivery of gases require a large, concentrated, prolonged administration to reach a therapeutic dose. We have shown that DSPC (particularly with PEG5000, and to a lower extent, with PEG2000) can make stable, pure NGMBs through shaking. A continuous sonication system is one way to produce large quantities of bubbles. In this report, we have shown that the sonication/centrifugation method can be applied to Xe as well, and larger concentrations of bubbles can be produced with relative ease. Our protocol involves an open-to-atmosphere system, but one can also use a closed formulation line.

The present disclosure shows that the delivery vehicles for various noble gases can be generalized. One exemplary study indicates that a long-chained lipid like DBPC can stabilize sub-10 μm MBs for gases with varying properties like Ar and He, with two different commonly used PEG stabilizers. Argon is marginally less water-soluble than Xe, but significantly smaller; He is small enough to diffuse out of most membranes, while being sparingly water-soluble. Over time, the small size of He seems to enable its outward dissolution.

Experimental

Materials

DSPC and DBPC were purchased from Avanti Polar Lipids, Inc. (Alabaster, Ala.), while DSPE-PEG2000 and DSPE-PEG5000 were obtained from NOF America Corporation (White Plains, N.Y.). Phosphate buffered saline (PBS), chloroform, and fetal bovine serum (FBS) were purchased from Fisher Scientific (Pittsburgh, Pa.).

Formulation of Microbubbles

For stock solutions, primary phospholipids (DSPC or DBPC) and lipopolymer (DSPE-PEG2000 or DSPE-PEG5000) are dissolved together in chloroform in a 9:1 molar ratio. The chloroform is then evaporated in a fume hood, followed by vacuum overnight. The dried lipid-polymer film is then resuspended in PBS so as to make a final concentration of 10 mg/mL, and stirred for 45 min at 75° C. (for DSPC) or 90° C. (for DBPC). Next, the solutions are allowed to cool down to RT.

Subsequently, 1-1.3 mL of the lipid-PEG solution is taken into a 2 mL sealed vial, and gas (Xe or Ar or He) is bubbled into the vial through the rubber septa for 3-5 min.

For preparing MBs via shaking, the headspace of the 2 mL vial containing 1.3 mL solution is purged with the respective gas; the vial is then agitated at ˜4300 Hz in a Vialmix® (Bristol-Myers Squibb Medical Imaging Inc.) shaker for 45 s. The vial is left standing for ˜2 min, and bubbles are collected from the middle of the solution for counting. Similarly, bubbles are also collected after storage at 4° C., as required, after inverting the vial a few times by hand.

For preparing MBs via sonication, 1 mL of the gas-saturated lipid-PEG solution is transferred to a 2 mL microcentrifuge tube. The solution is then immediately probe-sonicated using a ⅛″ tip (Branson Digital Sonifier 250) for 20 s, with the tip being moved slowly along the height of the liquid during the sonication cycle to ensure uniform energy dissipation. The tube is then kept on ice for 5 min. Following this, the MB dispersion is centrifuged at 250 g for 2.5 min; majority of the infranatant is discarded and replaced with the respective gas-infused PBS (gas bubbled into PBS for 1-2 min). After a subsequent washing step, the resulting MBs in the tube are aspirated into a 1 mL plastic syringe with a 20-gauge needle. After ˜2-3 min, large MBs float to the top of the inverted syringe, and the smaller MBs are pushed out through the needle and collected into a vial. The headspace of the vial is purged with the respective gas, and it is stored at 4° C. for further use.

Microbubble Counting

10 μL of appropriately diluted MB solution is loaded into a haemocytometer (Hausser Scientific™ Levy™) and images are captured using a Zeiss upright microscope. 5-7 images of each sample are then analyzed using ImageJ (NIH) to determine the size distribution and concentration of all particles 800 nm or greater.

Xe Detection Via Cryptophane Quenching

A water soluble cryptophane, tris(triazole propionic acid) cryptophane-A derivative (TTPC) is prepared as described before. 2 μL or 10 μL of as-prepared XeMBs are added to 90 μL of 30 μM TTPC to a final volume of 100 μL in PBS. After 2 min, the mixture is transferred into a quartz cuvette, and the fluorescence intensity is measured at 280 nm and 300 nm excitation and emission wavelengths respectively, using a Varian Cary Eclipse fluorescence spectrophotometer, at a PMT voltage of 1000 V. For calibration, Xe gas is bubbled into PBS for 15 min; different volumes of Xe-saturated PBS is then mixed with TTPC for 2 min, and their fluorescence is measured. With the solubility of Xe in water being 0.097 mL/mL at RT, a calibration curve of Xe concentration vs. fluorescence intensity was developed. The average peak intensity value at 315 nm (from at least 3 samples) is used to calculate the quantity of Xe present in the respective solutions.

Gas Chromatography-Mass Spectrometry

100 μL of as-made gas bubbles are transferred into an empty, 2 mL serum vial, followed by sealing the vial with a rubber septa. The vials are then bath-sonicated for 1 min, following which, ˜10 μL of the gas in the vial headspace is withdrawn using a GC-syringe, followed by immediate injection into the GC-MS system for analysis. For calibration, fixed volumes of gas (He, Ar, or Xe) are injected into sealed 2 mL vials, and the same procedure is utilized for measuring MS intensity. A calibration curve based on the known volumes of gas injected vs the MS intensity is developed, from which the amount of gas in the MB solution headspace can be determined.

Ultrasound Contrast Imaging and Analysis

For ultrasound imaging studies, a commercial Zonare scanner images the MBs in both B-mode and Contrast mode using a L14/5 transducer immersed in a 37° C. water bath. The frequency and mechanical index (MI) used are 3.4 MHz and 0.12, unless specified otherwise. MBs are diluted in PBS or 50% FBS to a final volume of 0.3 mL in a plastic transfer pipet, and are incubated at 37° C. for 1 min. The pipet is then placed in front of the transducer, and 5 min videos are recorded. Analysis of recorded videos is performed using a custom code written in MATLAB (Mathworks, Inc.). Each video is broken down into still images based on the recording frames per second rate. An ROI is then manually selected to cover the area containing the bubbles. The images are converted into grayscale, and the mean pixel intensity (total intensity/area of ROI) is calculated for each frame over time.

In Vivo Ultrasound Imaging

The animal protocol using adult male athymic nude mice for this study was approved by the Institutional Animal Care & Use Committee (IACUC), and has been described in a previous study. Anesthetization of mice is carried out with isoflurane inhalation using an isoflurane vaporizer (VetEquip Inc., Livermore, Calif., USA). Imaging is performed under anesthesia (isoflurane vapor (1-2%) mixed with oxygen (100-200 mL/min)). 30 μL of XeMBs (˜4×10⁹) is injected retro-orbitally. Non-linear contrast enhanced and B-mode ultrasound videos of either the heart or the kidney are acquired using a 13-24 MHz MS250 transducer (Vevo LAZR, FUJIFILM VisualSonics, Toronto, ON, Canada). Imaging is optimized and carried out at 18 MHz at 10% transmit power. Analysis of contrast recordings is performed using proprietary Vevo CQ software.

Abbreviations

MB: Microbubbles; NG: Noble Gas; PFC: Perfluorocarbons; DPPC: 1,2-dipalmitoyl-sn-glycero-3-phosphocholine; DSPC: 1,2-distearoyl-sn-glycero-3-phosphocholine; DBPC: 1,2-dibehenoyl-sn-glycero-3-phosphocholine; DSPE-PEG2000: 1,2-distearoyl-sn-glycero-3-phosphoethanolamine-N-[amino(polyethyleneglycol)-2000] (ammonium salt); DSPE-PEG5000: 1,2-distearoyl-sn-glycero-3-phosphoethanolamine-N-[methoxy(polyethylene glycol)-5000] (ammonium salt).

Additional Disclosure—Multivariable Dependence of Acoustic Contrast of Fluorocarbon and Xenon Microbubbles Under Flow

Microbubbles (MBs), are 1-10 μm gas cores stabilized by phospholipids, polymers, or proteins; they are excellent ultrasound contrast agents owing to their compressible gas cores and resonance properties at clinical ultrasound frequencies. Additionally, MBs have been widely studied for their use in drug/gene delivery, photoacoustics, cancer treatment, and more recently, as carriers of therapeutic gases such as oxygen and xenon.

Several prior publications have reported on the stability and echogenicity of MBs. Studies have shown that the composition and properties of the shell have substantial effect on the persistence and acoustic response of microbubbles. For example, increasing the acyl chain length of the phospholipid that stabilizes MBs leads to improved monolayer packing and a stiffer shell, causing slower gas dissolution and increased in vivo persistence. More recently, a strong inverse relationship between the shell stiffness and the microbubble echogenicity has been demonstrated by combining a recombinant protein and Pluronic copolymer for stabilizing MBs. Also studies have reported the effect of the core gas on the stability and echogenicity of MBs, wherein the increase in inherent solubility of the core gas in aqueous media causes a propensity towards easier dissolution of the bubble in a multigas environment. Most of these studies that correlate the composition of the MB shell and gas core to their stability and echogenicity, however, have been performed under static conditions despite the fact that MBs are almost always used under flow.

The contrast signal generated by MBs as well as their stability under flow can be affected simultaneously by a complex array of factors including bubble core gas composition, shell composition, bubble concentration, and bubble size. From the physiological standpoint, blood flow rate, blood vessel diameter, blockage, and shear, along with vascularization could potentially affect the stability and contrast properties of MBs under flow. During imaging, the mechanical index, positioning and dimensions of the transducer as well as imaging frame rates, and post-processing will affect the final contrast enhancement data. Many of these factors are inter-dependent in their effects, making modelling and observing their simultaneous interplay a non-trivial task. While a few prior studies have investigated the contrast properties of MBs under flow, these have focused on the temporal aspect of contrast, such as the persistence of bubble circulation for one particular type of MB under one set of imaging parameters. Others have investigated the change in bubble backscatter/contrast as a function of one or two parameters such as low acoustic pressures at two high flowrates in vivo for velocimetry applications, the rate of perfusion in vasculature in vivo, or the rate of bubble replenishment and transmit power in vitro, all tested for one bubble composition at a time. However, to understand mechanistic details about contrast enhancement as well as gas delivery in physiological experimental conditions, it is essential to study the echogenicity of MBs under flow, while examining the simultaneous effects of shell, flow, and imaging parameters.

In this study, we investigate the effects of composition of the microbubble shell as well as ultrasound scanning parameters on the contrast properties of MBs under controlled flow conditions in a tissue-mimicking phantom. We aim to study MB behavior of both diagnostic and therapeutic gases. For the former, we use perfluorobutane (PFC), which like other water-insoluble fluorinated gases, is commonly used as the core gas for commercially available and widely studied MBs intended for imaging. For the latter, we use xenon (Xe), which is one of the most promising therapeutic gases for MB-based delivery for ischemia-reperfusion injury. Xe gas is known for antiapoptotic properties in a variety of cases involving hypoxia ischemia injuries, and has been shown to have neuroprotective, cardioprotective, and nephroprotective effects in vivo. Our recent work has shown the feasibility of encapsulating the water-soluble Xe inside stable and echogenic MBs. However, to our best knowledge, few studies have reported on the echogenic properties of water-soluble therapeutic gas-based microbubbles under flow.

We observed the initial and final states of microbubble contrast as well as the evolution of contrast signal along the direction of flow as represented by the contrast gradient CG (spatial rate of decay of signal) and the contrast peak CP (where the bubble population shows maximum echogenicity). We hypothesize that the CG from exposure to ultrasound along the length of the tube/transducer will be affected both by the insonation parameters and the acoustic response of the bubble determined by their dynamic stability. We vary both mechanical index (MI) and flowrate (F) for bubbles of different shell stiffnesses (S) and show a number of non-linear trends in ultrasound signal (US) for a specific core gas. Even small changes in any of these factors can produce significant changes in CG, which can be both a challenge to precise design of MBs and also an opportunity to extract a large amount of data from imaging which would otherwise be overlooked. These trends can thus potentially be used to understand both bubble material properties and physiological flow conditions from reverse-engineering the non-linear bubble contrast.

Materials and Methods

Materials. DPPC (1,2-dipalmitoyl-sn-glycero-3-phosphocholine), DSPC (1,2-distearoyl-sn-glycero-3-phosphocholine), and DBPC (1,2-dibehenoyl-sn-glycero-3-phosphocholine) were purchased from Avanti Polar Lipids, Inc. (Alabaster, Ala.), while DSPE-PEG2000 (1,2-distearoyl-sn-glycero-3-phosphoethanolamine-N-[methoxy(polyethylene glycol)-2000] (ammonium salt)) was obtained from NOF America Corporation (White Plains, N.Y.). Phosphate buffered saline (PBS) and chloroform were purchased from Fisher Scientific (Branchburg, N.J.). Perfluorobutane and xenon were purchased form FluoroMed LP (Round Rock, Tex.) and Airgas, Inc. (Radnor, Pa.) respectively. Silicone (Ecoflex-00-30) was purchased from Smooth-On, Inc (Macungie, Pa.).

Formulation of microbubbles. To formulate perfluorobutane (PFB) MBs, To formulate MBs used in this study, a sonication/differential centrifugation method is used to obtain uniform MBs from a polydisperse population using an approach described earlier (Blum et al. 2017). Primary phospholipids (DSPC or DBPC) and lipopolymer (DSPE-PEG2000) are dissolved together in chloroform in a 9:1 molar ratio. The solvent is then evaporated in a fume hood, followed by vacuum overnight. The dried lipid-polymer film is then resuspended in PBS so as to make a final concentration of 1.5 mg/mL and stirred for 45 min at 75° C. (for DPPC and DSPC) or 90° C. (for DBPC). Next, the solutions are allowed to cool down to RT. Subsequently, PFB gas is bubbled through 1 mL of the lipid-PEG solution for 2-3 min. in a 2 mL microcentrifuge tube. The solution is then immediately probe-sonicated using a ⅛″ tip (Branson Digital Sonifier 250 (Branson Ultrasonics, Danbury, Conn.)) at 10% power for 10 s, with the tip being moved slowly along the height of the liquid during the sonication cycle to ensure uniform energy dissipation. The tube is then kept on ice for 1 min. Following this, the MB dispersion is centrifuged at 250 g for 2.5 min; majority of the infranatant is discarded and replaced with the respective gas-infused PBS (PFB bubbled into PBS for 1-2 min). After a subsequent washing step, the resulting MBs in the tube are aspirated into a 1 mL plastic syringe with a 20-gauge needle. After ˜2-3 min, large MBs float to the top of the inverted syringe, and the smaller MBs are pushed out through the needle and collected into a vial.

For xenon (Xe) MBs, DBPC and DSPE-PEG2000 (9:1) are directly mixed in PBS at a concentration of 10 mg/mL 90° C. Xe gas is bubbled into a 5-10 mL stock solution for 3-5 min in a 20 mL glass vial, followed by sonication throughout the height of the liquid using a ½″ sonicator tip at 70% power. Following this, the vial is kept on ice for 1 min, and the bubbles are withdrawn and locked into 3 mL syringes, which are then centrifuged at 300 g for 3 min to obtain a concentrated Xe MB sample. For both PFB and Xe MBs, the headspace of the storage vial is purged with the respective gas and stored at 4° C. for further use. The size and concentration of MBs are determined using a hemocytometer (Hausser Scientific, Horsham, Pa.) by processing 5-7 images from different samples using ImageJ (NIH), where counting of particles was carried out automatically by the software. The size distribution is determined by automatically counting the number of bubbles using ImageJ and by subsequent binning of the population into 20 bins from 0-20 μm. Particles lower than 0.8 μm in diameter were not considered to eliminate metastable nanobubbles or liposomes in the count.

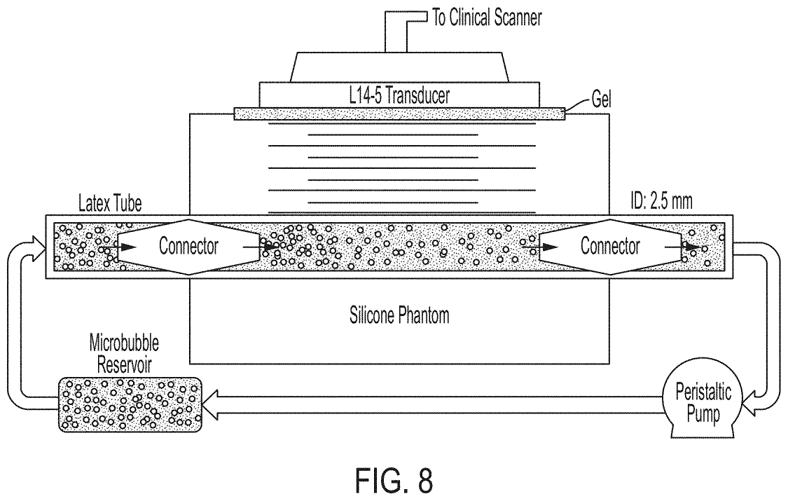

Phantom. A latex tube with ˜2.5 mm internal diameter and wall thickness of ˜0.85 mm is attached longitudinally in the middle of a rectangular container and connected to opposite walls using a venturi-connector, which further makes a circuit going through a peristaltic pump and a reservoir containing the bubbles (FIG. 8). The container is filled with silicone (which is then cured), leaving the tube embedded in the gel. An ultrasound transducer is placed longitudinally along the axis of the tube for imaging, which corresponds to a length ˜5.5 cm of the tube being imaged at a time. For static imaging, MBs are diluted in a plastic transfer pipet and place along the transverse axis of the transducer, both immersed in a water bath.

Ultrasound imaging and image analysis. After adjusting the pump to the desired flowrate setting and scanner to the desire MI, flow was equilibrated for ˜10 s, followed by recording of a 3s video at 15 fps. A L14-5 transducer connected to a Zonare ZS3 clinical scanner (Mindray North America, Mahwah, N.J.) is used for all experiments in this study. The Zonare scanner uses zone sonography for capturing and processing images; unlike conventional scanners, it does not acquire echoes one line at a time which adds up to a high pulse-echo round-trip time. Imaging is instead carried out by transmitting around 10 broad plane wave beams per frame and corresponding data from each zone is processed. In this study, we scanned the MBs at a center frequency of 3.4 MHz in non-linear contrast mode. This is repeated for all flow and MI conditions for different MB samples. A custom Interactive Data Language (IDL) code (L3Harris Geospatial, Broomfield, Colo.) code is used to carry out image analysis. The videos containing the log-compressed data, as generated by the scanner, are converted into grayscale, and an ROI outlining the lumen is drawn in the first image and copied on to the remaining frames. Each ROI is subdivided up into 250 sections from inlet to outlet, and the mean contrast brightness for each section is calculated to provide a contrast signal graph over the length of the ROI. The signal values of each section are then averaged over the number of frames to provide the final signal v/s direction of flow plots for each video.

Static Imaging. Under static conditions, two protocols are used for imaging. First, imaging is carried out in the same phantom, but with the flow stopped once bubble flow equilibrated inside the tube. Videos were recorded immediately after the flow is stopped. Second, the same procedure is followed as above, except that MBs are placed in a polyethylene transfer pipet bulb in a water bath. The transducer is placed orthogonally in front of it, and videos are recorded immediately after ultrasound is turned on. Images are analyzed in the same manner as described above.

Results

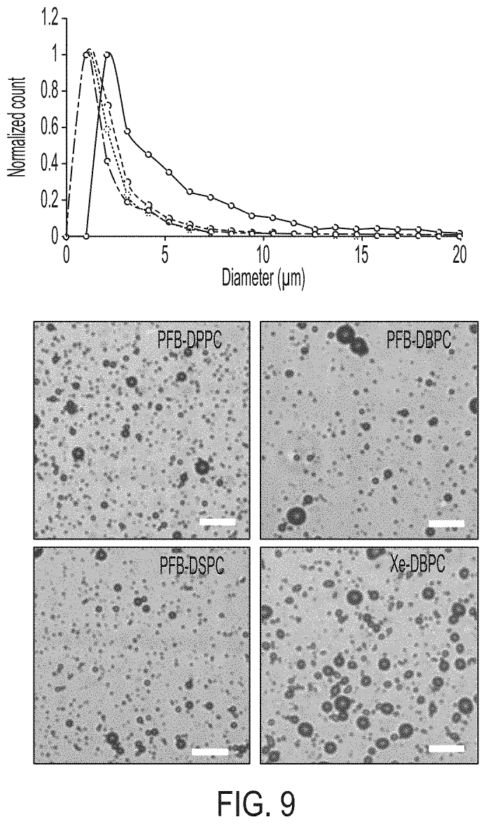

Microbubble characterization. The microbubbles formulated here have a perfluorobutane (PFB) core and a shell comprising primarily of either DPPC (C16:0), DSPC (C18:0), or DBPC (C22:0), which are phospholipids differing only in their acyl chain length. Size distribution of PFB MBs, as shown in FIG. 9, provide a sufficiently uniform population with mean diameters of 3.2±0.45 μm (DPPC), 3±0.4 μm (DSPC), and 3.12±0.1 μm (DBPC), with the errors representing 1 standard deviation. Xe MBs produced have a wider distribution with a mean size of 6.5±0.6 μm. Maintaining a uniform size distribution helps PFB MBs attain better acoustic imaging efficiency (Segers et al. 2018), whereas it is not as essential for therapeutic MBs with gases such as oxygen, nitric oxide, or xenon since the primary purpose of such MBs is to deliver their gas payload. Furthermore, it is desirable that therapeutic MBs are larger in size while being less than 10 μm to enhance payload capacity.

Perfluorocarbon microbubbles under flow. To establish a relationship between the contrast properties of MBs and various parameters including MB shell composition, flow rate and MI under flow, we observe the ultrasound signal along the length of a transducer positioned on MBs flowing in an acoustically transparent tube embedded in a tissue-mimicking silicone phantom (FIG. 8). We use different flowrates, with pulsatile flow using a peristaltic pump; under the example flowrates, we subject the MBs to a range of clinically relevant mechanical indices including 0.12, 0.18, 0.31, 0.5, and 0.8. The MB concentration for the experiments is chosen such that the most echogenic DPPC MBs do not saturate the initial contrast at MI of 0.8, while the least echogenic DBPC MB initial contrast is visible at MI of 0.12.

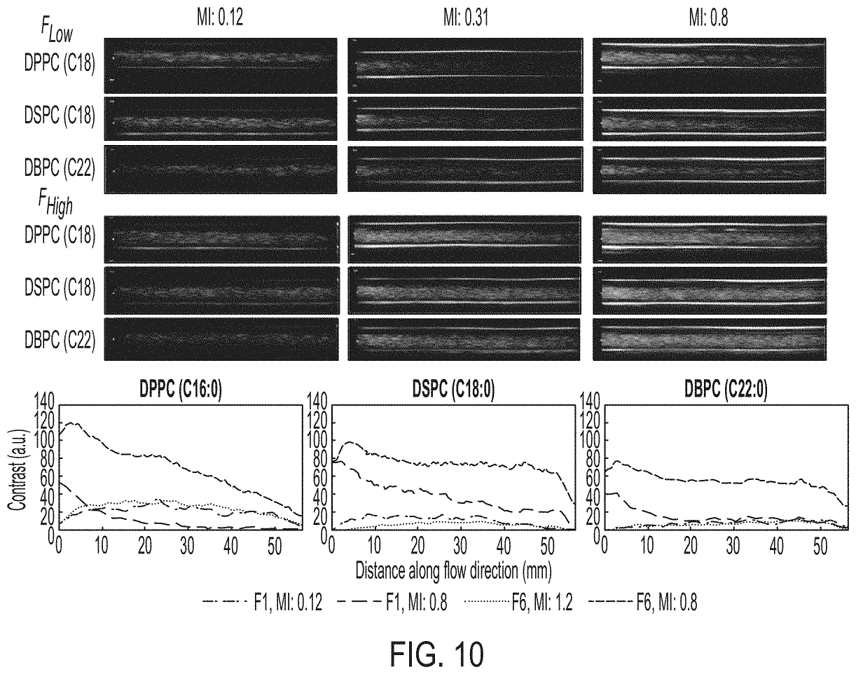

FIG. 10 shows the acoustic signal of PFB MBs at the upper and lower bounds of the flowrate conditions under 3 different MIs spanning the range. The results show that differences in insonation is not only in the image contrast, but also the contrast gradient (decay rate of signal, CG) along the direction of MB flow in the tube. We examine the effect of three pertinent input variables in our system—mechanical index, flow rate, and shell stiffness—in further detail on the CG along the direction of flow.

Effect of mechanical index (MI). MI is the ratio of peak negative pressure and the square root of the center frequency. Since the incident frequency is kept constant in this study at 3.4 MHz, the MI becomes an indicator of the incident pressure applied by the transducer. The lowest and highest MIs used are 0.12 and 0.8, corresponding to approximately 220 kPa and 1,475 kPa, respectively.

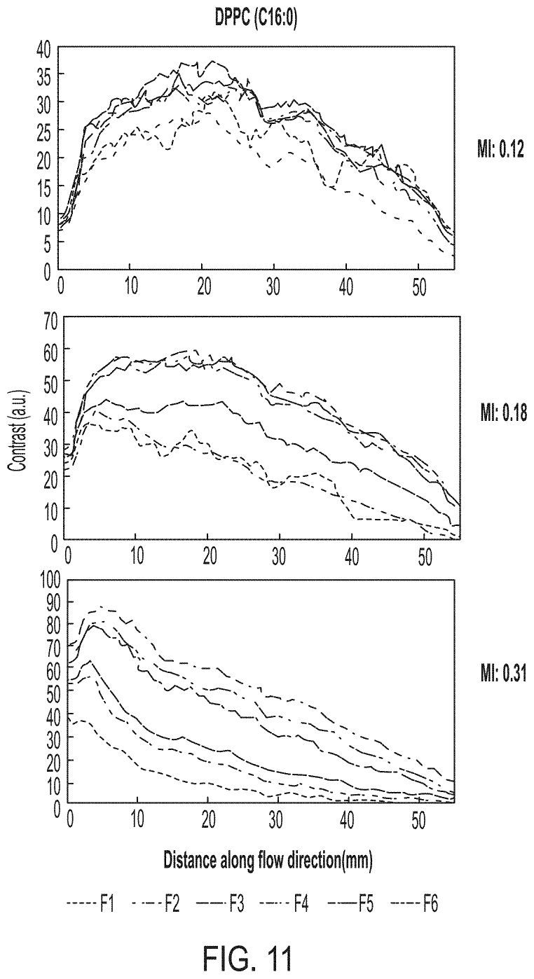

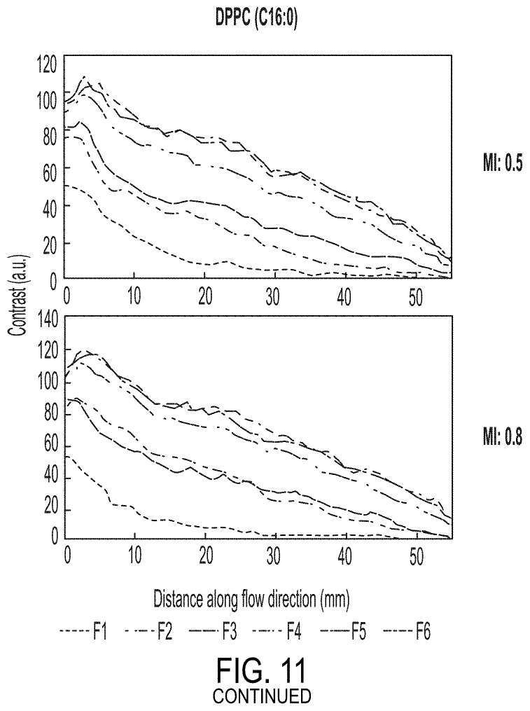

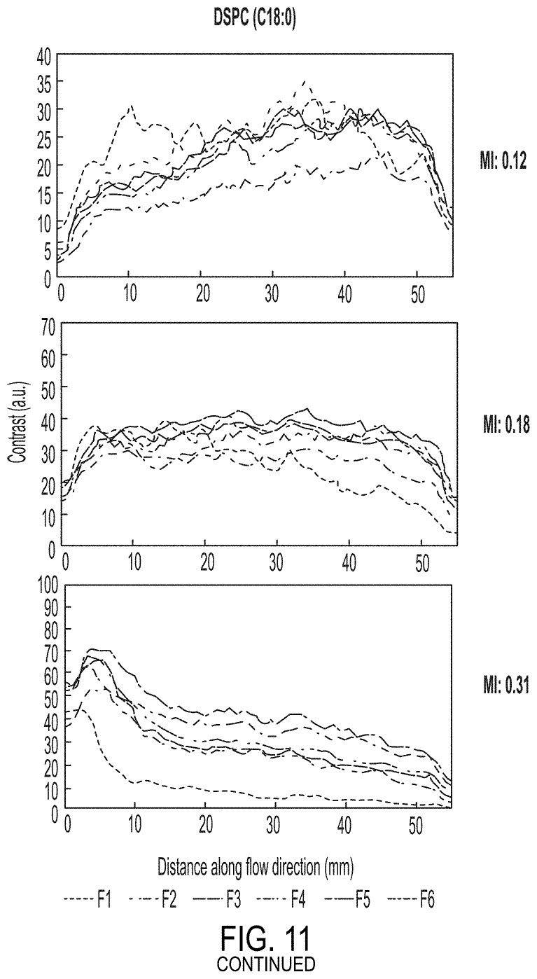

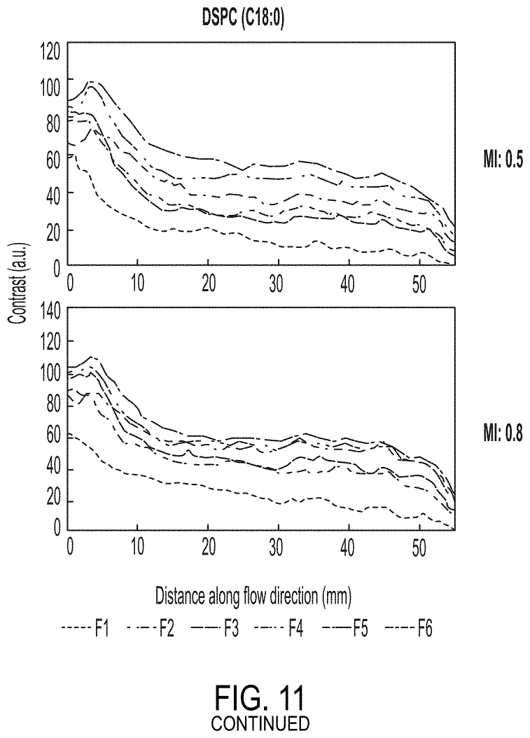

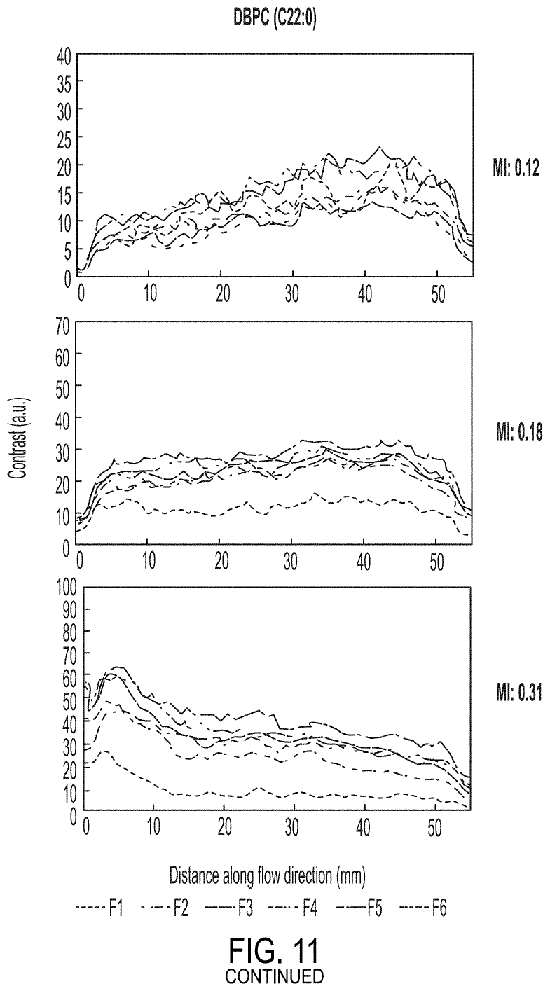

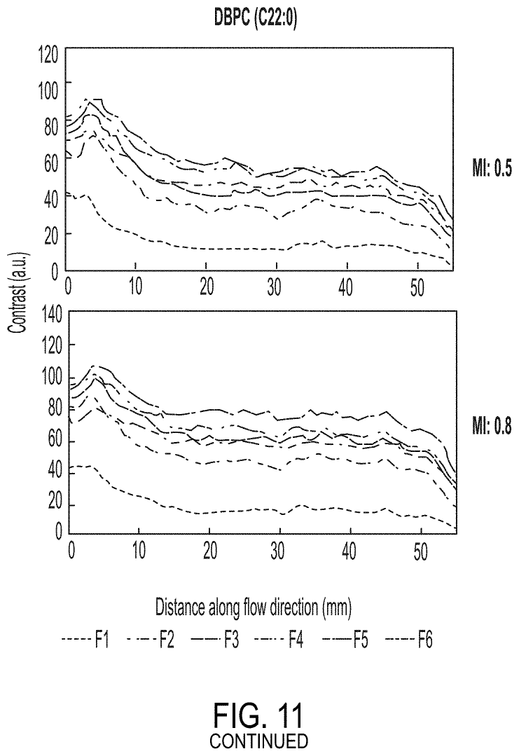

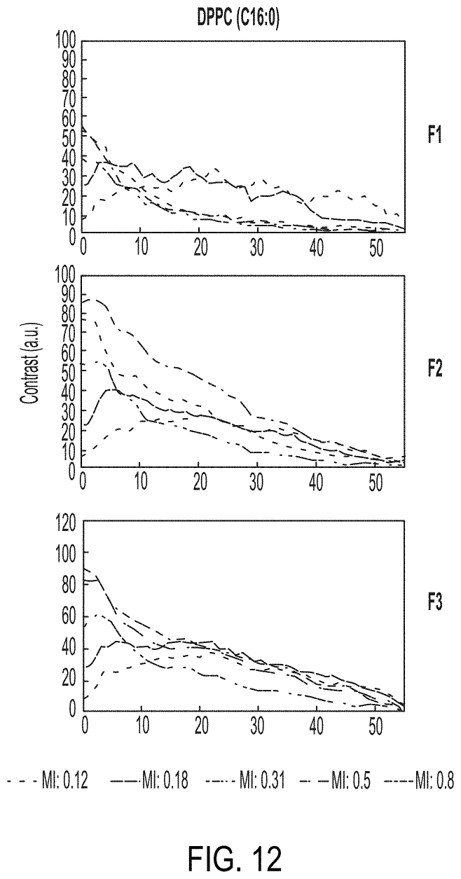

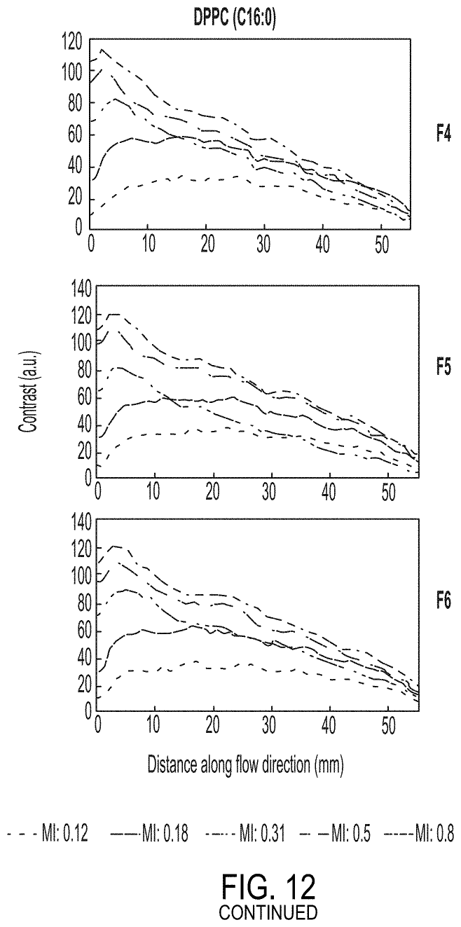

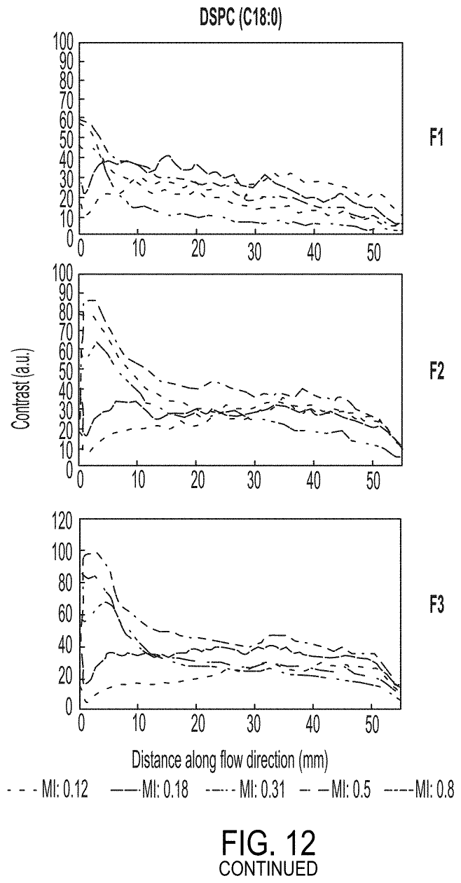

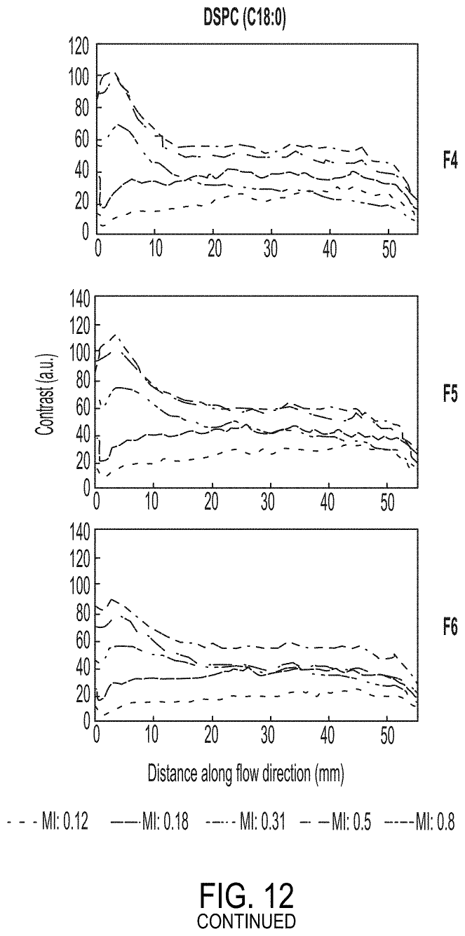

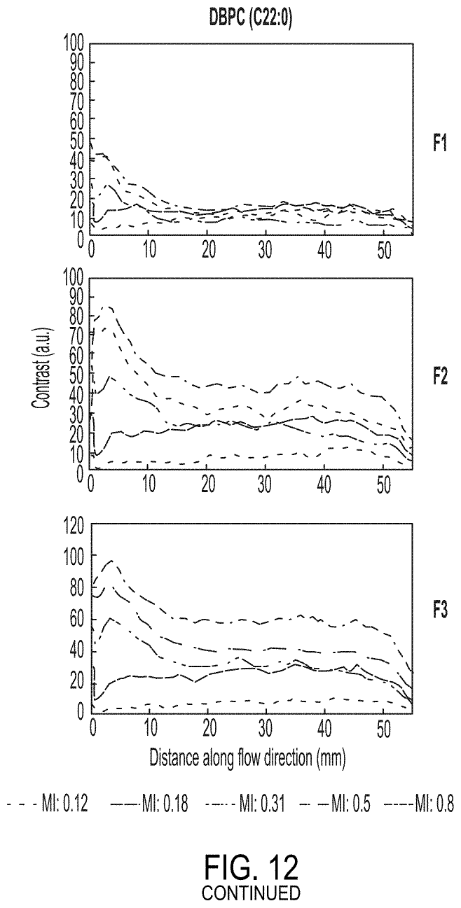

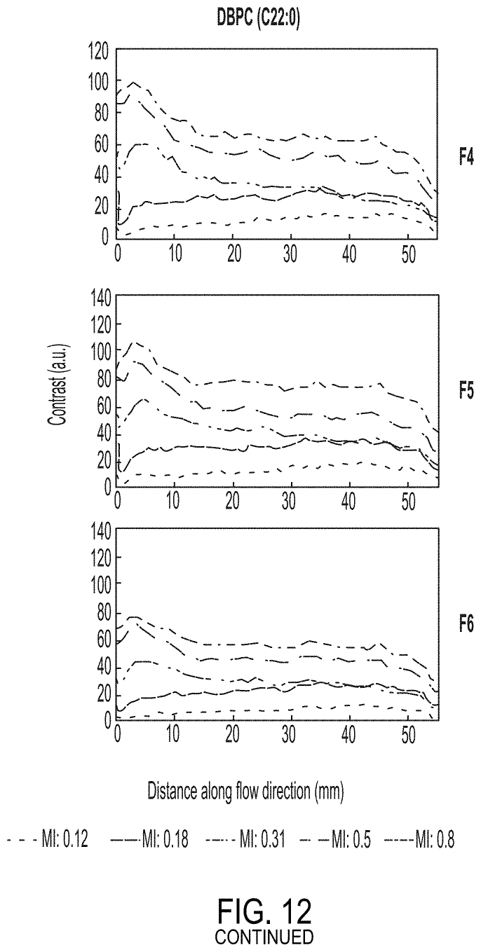

Quantifying the brightness data for the extreme flowrate and MI conditions in FIG. 10, we see an increase of non-linear contrast when increasing MI from 0.12 to 0.8. (A complete analysis of imaging under all flowrate, MI, and stiffness conditions is provided in FIGS. 11 and 12). At high MI, with increase in flowrate, we also observe a slightly higher signal near the inlet point for the same bubble bulk concentration. This signal difference could stem from a variety of factors: a) the increase in the amount of non-linear to linear scattering due to higher incident pressure, b) the venturi-based geometry of the inlet allowing a high flowrate causing the torrent of incoming bubbles to spread over a wider transverse area in the tube, and c) the fixed frame rate of image capture where a higher number of MBs pass through the same field of view per unit time for a higher flowrate. With flow, an MI of 0.8 proceeds to bubble destruction/dissolution for majority of the MBs towards the exit end of the tube length, resulting in reduced signal.

Another important variable is the contrast peak (CP) which indicates the position of the tube from the inlet where contrast value reaches a maximum. With increasing MI, the CP shifts left towards the tube inlet. This shift is likely due to the greater bubble dissolution and destruction that characterizes increasingly earlier sections of the tube at high MIs.

Effect of flowrate. The velocity of MB infusion into a blood vessel or perfusion into vasculature dictates both the shear that the bubbles experience (when flowrate is very high or turbulent) and the time under imaging ultrasound exposure per unit length of flow (t_{US}). First, FIGS. 10 and 12 show that, irrespective of F, contrast rises to a slight peak before leveling off towards the outlet. The reason the peak is located slightly downstream from the field-of-view inlet, we believe, is due to the venturi-based geometry and possibly due to increasing t_{US} along the flow direction. With increase in F, t_{US} decreases, both allowing for less ultrasound pressure exposure and for faster replenishment of bubble population. The CP shifts marginally right away from the tube field-of-view inlet as F goes up, which is especially noticeable in the low F regime. Because of the decrease in residence time under ultrasound with increasing flowrate, the distance travelled by the bubbles to reach a contrast peak may increase marginally. Due to the decrease in t_{US} with rise in F, the CG becomes less steep; this effect is more pronounced when MI is high (high pressure pressure), wherein reduction in the number of bubbles due to collapse dictates CG over the tube.

Effect of shell composition. MBs studied in this work are stabilized by phospholipids with fully saturated alkyl tails of different lengths. The images and data in FIGS. 10, 11, and 12 show a decrease in initial MB signal from C16 to C22 near the flow inlet, consistent with the expected increase in the resistance of C22 MBs to gas dissolution and MB collapse, as discussed later. Additionally, we also observe a higher residual MB signal towards the outlet from C22 than from C16, indicating significantly less bubble destruction for C22. Combining these two observations, we see that CG along the direction of flow decreases in general from C16 to C22. This effect is more evident at high MIs and low F, due to the quicker destruction of C16 MBs. Conversely, the CP shifts slightly left towards the tube entrance from C22 to C₁₆ and is pronounced for low MI and high F conditions. This trend can be explained by the shell undergoing lipid monolayer disruption/shedding thereby affecting their rigidity and increasing MB oscillation. For stiffer shells, the cycle of buckling, bending, and collapse of the bubble, as reported in previous studies will be more resistant to ultrasound induced compressive stress.

Considering all of these results together, it is clear that the CG and CP depend on more than one factor simultaneously. A brief summary of the multiple effects of F, MI, and S is provided in Table 2 as an empirical primer for flow properties of PFB MBs under flow.

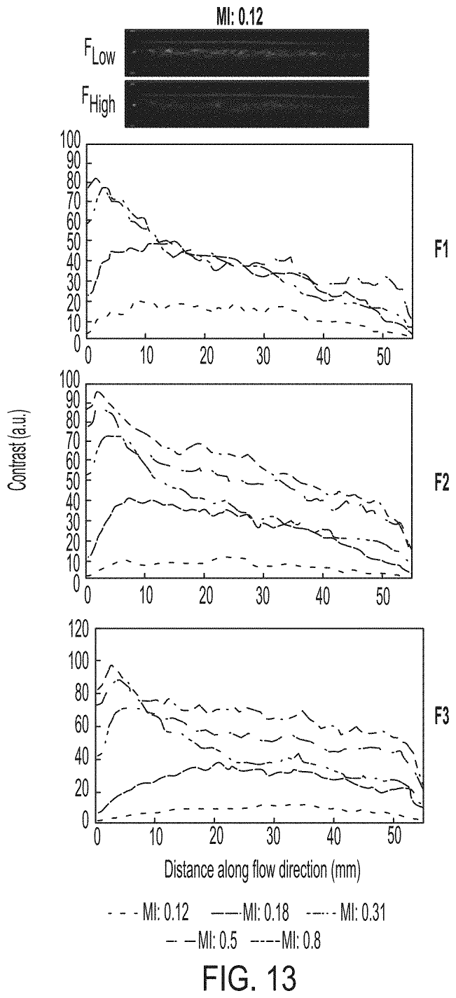

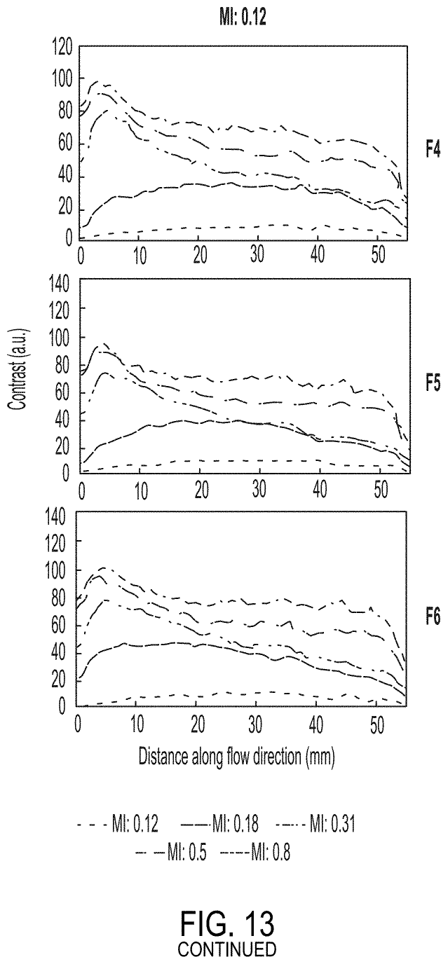

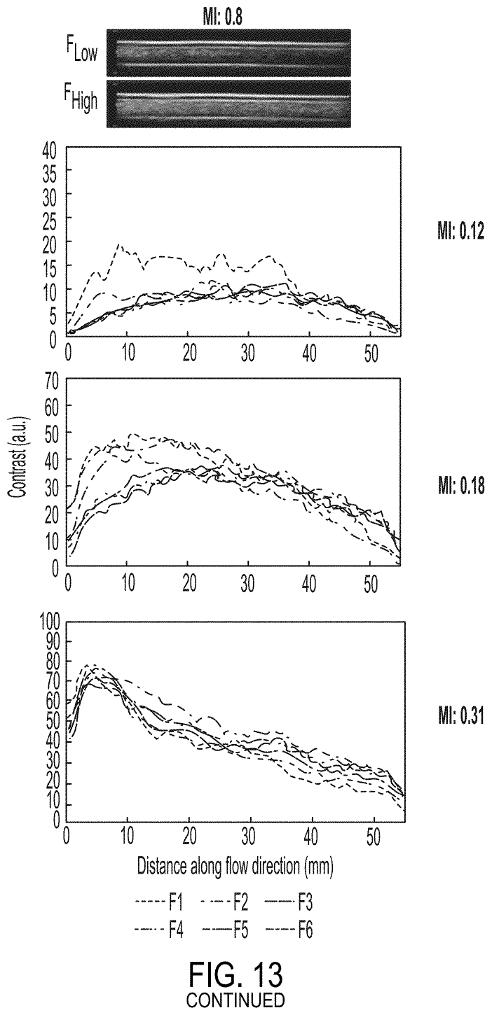

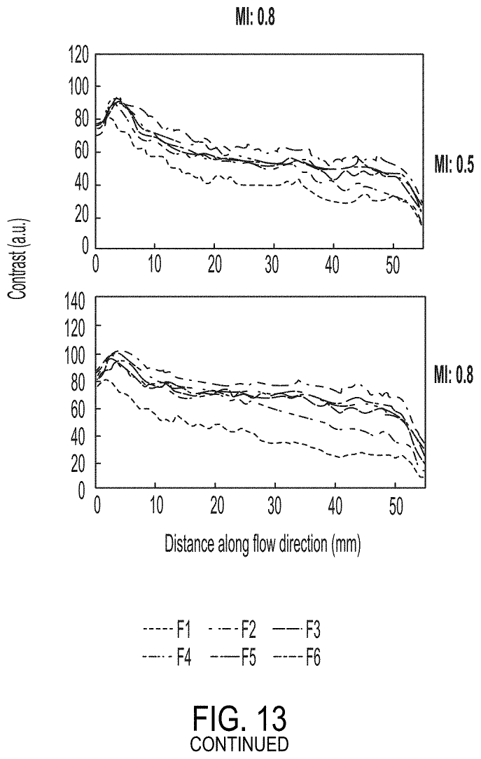

Xenon microbubbles under flow. We also investigate the potential effect of the core gas on the flow contrast characteristics of MBs. From FIG. 13, we see that DBPC-stabilized Xe MBs follow a similar overall trend for CG and CP as that of DBPC-stabilized PFB MBs. There are, however, some subtle differences between the two types of MBs. First, we find that we need approximately three times the concentration as that of PFB MBs to obtain similar mean contrast at the starting extreme F and MI conditions (F1 and MI_{0.12}). This need for a higher concentration stems from the inherently lower stability of MBs containing a water-soluble gas such as Xe due to a much higher chemical potential gradient between the bubble core and the aqueous phase. Second, we observe a higher initial signal for low F/high MI conditions than for PFB MBs. This high signal can be attributed to a higher Xe MB mean size causing an increased scattering; in fact, this is evidenced from the top-right panel of FIG. 13 where Xe MBs, owing to their larger size and increased buoyancy, concentrate towards the top of the tube, given enough time. Third, from MI_{0.18} onwards, the initial and mean MB signal suddenly increases, unlike for PFB MBs, where the rise in contrast with MI is more gradual. Previous models have predicted that water-soluble gases, unlike PFB, will see a high initial fall in gas content from inside a bubble followed by plateauing, assuming steady state conditions without ultrasound exposure. A sudden initial core shrinkage brought on by a threshold pressure (MI_{0.18}) could cause a cavitation induced signal increase.

Static imaging. We first image MB samples under static conditions in a polyethylene pipet bulb, surrounded by water, similar to imaging setups for bubble imaging in many studies. The concentration of PFB MBs is chosen such that it is just high enough not to cause signal saturation from the scanner, with DPPC-coated MBs used to calibrate the response. The concentration of Xe MBs is chosen so that the initial signal corresponds closely to that of the DBPC PFB MBs, since the shell material of Xe MBs is also composed of DBPC. FIG. 10 verifies the echogenicity of all the different MB samples at MI of 0.12. When MI is increased to high values such as 0.8, the MBs suffer dissolution/destruction within the short equilibration time. Next, we conduct static imaging using our flow phantom by simply turning off bubble flow (FIG. 10). While the ultrasound signal time decay trends are similar to that of the pipet bulby, the magnitude of the signal is markedly less, similar to magnitudes obtained in flow studies described previously. We find that even with 3-4 times the concentration used for imaging in a polyethylene tube, the mean signal, especially at low MI (0.12) is still considerably lower in the silicone phantom, as seen from FIGS. 10 and 11, likely due to the different levels of ultrasound attenuation by the different imaging phantoms.

Discussion

The biocompatible phospholipids used as a shell stabilizer for MBs in this study have been extensively reported in literature and form an ideal system to compare MB behavior as a function of shell stiffness since the only difference between the lipids is the length of their acyl chains. Additionally, we have shown that pure xenon microbubbles (Xe MBs) can be stabilized for long durations only by DBPC, while providing excellent echogenicity both in phantom and in vivo scenarios. To start with, we expect DPPC-coated MBs to allow for the generation of the highest contrast immediately after ultrasound application. Slightly higher DPPC contrast than DSPC and significantly higher contrast than DBPC near the tube inlet (under flow) and just after turning on ultrasound (static conditions) indicate, that in the short term and between the range of C16 to C22, low shell stiffness dominates bubble stability under ultrasound as the deciding factor for echogenicity.

Our results from ultrasound signal variation under flow validates our hypothesis that the microbubble contrast gradient along the direction of flow depends on the cumulative effect of external parameters and bubble chemistry. Broadly, this expectation is based on the assumption that higher incident pressure (MI) will lead to greater bubble oscillation/compression and cavitation phenomena to enhance the received acoustic signal, while at the same time accelerating gas efflux and MB destruction to produce a greater contrast gradient (i.e., signal reduction along the flow direction). Increasing flowrate (F) will reduce the total residence time under the ultrasound transducer as well as the ultrasound exposure time per unit length of flow (t_{US}), reducing bubble collapse while also reducing bubble oscillation time. A stiffer shell (S) on a bubble will cause a closely packed monolayer enhancing bubble stability and producing lower oscillation leading to lower acoustic contrast.

The effect of mechanical index on phospholipid-shelled MB destruction has been previously reported to be initiated via dissolution at 400-600 kPa, with bubble collapse starting around 800 kPa (Borden et al. 2005). In clinical practice, MIs in the range of 0.1-0.2 is often used in pediatric and arterial imaging. Theoretical predictions and experimental validation have shown that inertial cavitation of indigenous bubble nuclei around tissue without any contrast agent would not occur below an MI of approximately 0.7, which led to the incorporation of MI as a biosafety guideline for ultrasound imaging applications. However, it is likely that stable cavitation and gas dissolution may start at much lower acoustic powers, resulting in a reduction in signal as is shown by the data presented in this report (FIGS. 10, 11, and 12). It should be mentioned here that the signal at low MI is dominated by oscillation/compression of MBs, whereas high MI signals are likely a result of inertial cavitation. Also, an increase in incident pressure in the form of MI will increase MB backscatter and potentially cause non-linear propagation of the echo signal through the silicone, resulting in additional harmonic signals from the phantom itself and from the MBs. Furthermore, the change in applied MI is representative of a change in incident acoustic power; the exact values of MI, however, should not be taken as absolute parameters for bubble signal dependence. It has been shown that depending on the type of transducer and scanner, a wide variety of bubble brightness can be generated. Therefore, the results presented in this study provide a trend for a given scanning system where MI can be gradually changed to shed light on the effect of flow or bubble materials on the MB contrast behavior.

Interestingly, the implications from the results in this study based on the effect of flowrate in MB signal variation and signal acquisition along the flow direction seems to have been largely overlooked in literature. The change in not just the mean signal, but the CG and CP as well, especially in the high and low MI regimes respectively, as a function of flowrate, show that the position (field of view) and dimensions (length of field of view) of the transducer play a significant role in delineating point-to-point bubble contrast during flow.

The effect of shell composition on microbubble dynamics with and without ultrasound under static conditions have been the subject of numerous previous investigations. Prior studies based on modeling and experimental data have shown the dependence of bubble oscillation, cavitation, dissolution and collapse on the structure of the shell material, as described below. From C16 to C22, the van der Waals energy between lipid molecules is postulated to increase by almost 50% (Garg et al. 2013). The enhanced cohesion creates a tightly packed shell, reducing both MB compression and efflux of core gas into the surrounding medium. A modified Epstein-Plesset equation describes the rate of shrinking of a bubble with radius r due to gas dissolution in a steady state condition as:

$$- \frac{dr}{dt} = \frac{H}{r / {D}_{w} + {R}_{shell}} ( \frac{( 1 + 2 σ / {P}_{a} r ) - f}{1 + ( 4 σ / 3 {P}_{a} r )} ) \qquad ( 1 )$$

where H is the Ostwald coefficient representing the ratio of gas concentration in the aqueous medium to that in the gas phases, a is the bubble-water interfacial tension, Pa is the ambient pressure, and f is the ratio of the actual partial pressure of the diffusing species in the surrounding medium to that at saturation, and D_{w} is the air diffusivity in water. The dissolution rate of gas from a bubble (−dr/dt) depends inversely on the shell resistance (Rₛₕₑₗₗ) against gas permeation. The relationship between monolayer resistance (Rₛₕₑₗₗ) and the number of carbons in the alkyl chain of the monolayer species (n) is given by:

Rₛₕₑₗₗ=Cexp[(Eᵣₑₛ+E_{CH}_{₂}(n−1))/kT] (2)

where C is a frequency factor, Eᵣₑₛ is the residual activation energy that takes into account the headgroup region and terminal methyl group, E_{CH}_{₂} is the activation energy per additional methylene group, k is Boltzmann's constant, and Tis the temperature. Modelling and experimental results from the same study show that shell resistance increases about six times going from C16 to C22, whereas the dissolution rate goes down by almost half from C14 to C22. The latter study also showed a ⅓^{rd} decrease in bubble fragmentation ratio with increase in acyl chain length from C14 to C22.

Similarly, there have been multiple reports indicating that increased shell stiffness resulting from increased shell thickness, elasticity, shear modulus, and dilatational viscosity causes enhanced damping of the oscillating shell, thereby increasing the threshold resonance frequency or power as well as inertial cavitation threshold. This is an important factor in explaining the initial difference in contrast from C16 to C22 at high MIs.

As expected, DBPC MB persistence was the highest. However, when measured at 7 MHz (MI of 0.49), the initial in vivo contrast upon entering the imaging window was lowest for DPPC MBs. In contrast, our results show that DPPC always provides the highest contrast for MBs near the tube inlet. The discrepancy may arise from the different mass transfer gradients exerted on the circulating MBs between in vivo and the current in vitro conditions. It could also depend on the type of harmonics being used to activate the MBs and the corresponding harmonic response. For instance, we find a partial discrepancy with our results in a separate report, where a higher second harmonic non-linear pressure amplitude response for DSPC MBs was observed as compared to that of DPPC MBs. However, DPPC MBs elicited a significantly higher subharmonic response, even more so with increasing the incident acoustic pressure from 50 to 100 kPa. Others have attributed this enhancement to the increased probability of buckling for DPPC MBs due to easier shrinking of the gas core; in the buckling regime, as opposed to the elastic regime, compression-only behavior dominates, and the amount of subharmonic response is high. We should note that the DSPC v/s DPPC MB study is also not a direct comparison since the incident acoustic pressure were far lower than the ones used here.

Out of the two chief components comprising a microbubble, the effect of the shell on bubble dynamics has been traditionally examined much more than that of the effect of bubble core. These effects are likely to be most pronounced for water-soluble therapeutic gas cores, as opposed to the water-insoluble PFB. Previously, numerous studies reporting the formulation of oxygen bubbles, mostly stabilized by DSPC as the primary phospholipid, reported the spontaneous release of the core gas in desaturated media. Similarly, another study noted the lack of contrast from DSPC-coated pure xenon MBs in vivo. However, we have recently shown that MBs with a pure xenon core can have significant and persistent static and in vivo contrast under low Mis. Around the same time, another report showed the preparation of echogenic oxygen MBs for the first time. Both of these studies used DBPC (C22) as the primary phospholipid and found the shell packing sufficient to stabilize pure therapeutic gas. We choose Xe MBs for this study since we have shown that DBPC-stabilized Xe MBs are echogenic in a phantom in static conditions for several minutes. However, in spite of the relative stability of DBPC-Xe MBs, a 3-4× concentration is required to elicit a similar signal to that of PFB MBs, as described in the Results section, along with other previously unreported differences in CG and CP under specific conditions (FIG. 13). These results highlight the importance of conducting comparative investigation of bubble dynamics under flow of increasingly researched MBs with a therapeutic gas core such as oxygen, xenon, and nitric oxide.

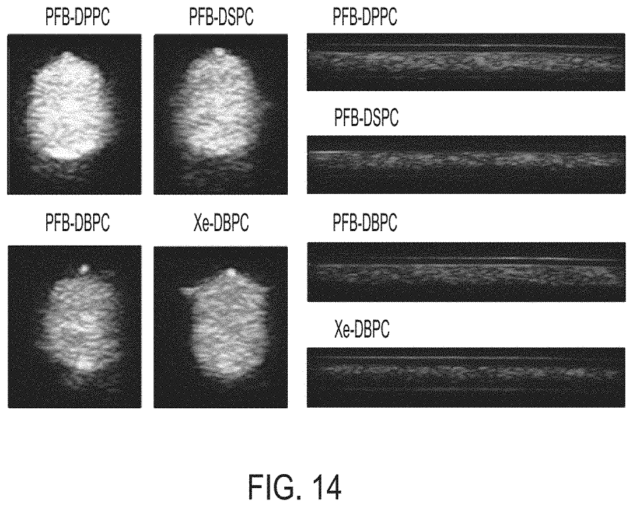

Lastly, it is often the case that expected behavior of contrast agents under flow in vivo is often extrapolated from their performance under static conditions, with insufficient consideration to the imaging system or eventual flow conditions. For instance, the attenuation coefficient at 5 MHz for the silicone phantom material used in this study is ˜15.5 dB/cm, while for polypropylene (material of the pipet bulb used for housing bubbles as well), it is ˜5.1 dB/cm. Additionally, in the polypropylene bulb system, water (with negligible attenuation) separates the phantom and the transducer, unlike in the silicone phantom system. These material and imaging parameters result in a marked difference in the magnitude of acquired MB signal between the two systems (FIG. 14). It is noteworthy in the context of this study that the MB signal time decay is similar for both static systems while not always corresponding to MB signal distance decay under flow. The slopes over time appear to be similar for C16 to C22, except for Xe MBs, where the inherent relative instability of the core leads to rapid bubble dissolution. This could be partially explained by potential uncontrolled replenishment where bubbles from the surrounding area can slowly occupy the field of view. Importantly, the signal decay slope represents a time of up to 3 min, whereas during flow, the signal variation is recorded with each MB receiving ˜1.7-14 s under ultrasound depending on flowrate. The data recorded under flow, therefore, allows us to resolve rapid contrast changes of an MB population along the flow direction. This increase in temporal resolution of signal of a population of bubbles using a straightforward setup like the one used in this study is something that is not easily achievable under stagnant conditions. Such an approach could be useful in bubble design, dosage, and imaging parameter considerations under clinical settings.

Conclusion

This study provides a rigorous demonstration of contrast enhancement by MBs under flow as a function of material properties like shell stiffness and core gas as well as ultrasound incident pressure (as represented by MI), and flowrate. While there is an enormous body of knowledge on single bubble and bubble population stability, oscillation, and cavitation properties under static conditions, there is a surprising dearth of literature on the potentially different behavior of the same bubbles under well-controlled flow conditions. Our findings suggest that the positioning and dimensions of ultrasound exposure (represented by the imaging transducer) can have a significant effect in changing the output signal, which can be used in conjunction with other known parameters such as MI, MB shell and core composition to potentially estimate the extent of flow, shear and related physiological properties. Additionally, we show the persistent contrast from flow imaging of a therapeutic gas (xenon) MB population for the first time. The goal of this study is to provide an empirical framework by which measurement systems with greater sensitivity than those in most current practice can potentially capture and interpret contrast agent flow profiles.

Additional Disclosure—Ultrasound-Guided Particle Delivery To Provide Protection

Traumatic brain injury (TBI) is associated with high mortality and morbidity in children and adults. Unfortunately, there is no effective therapy for treatment of TBI in the acute setting. Rodent studies have shown that xenon, a well-known anesthetic gas, can be neuroprotective when administered post TBI. However, gas inhalation therapy, the approach typically used for administering xenon, is expensive, inconvenient, and fraught with systemic side effects. Therapeutic delivery to the brain is minimal, with much of the inhaled gas cleared by the lungs. To bridge major gaps in clinical care and redefine cerebral delivery of xenon, this study introduces a novel xenon delivery technique utilizing microbubbles, in which high impulse ultrasound signal is used for targeted cerebral release of xenon. Briefly, an ultrasound pulse is applied along the carotid artery at the level of the neck upon intravenous injection of xenon microbubbles (XeMBs) resulting in release of xenon from microbubbles into the brain. This delivery technique employs a hand-held, portable ultrasound system that could be adopted in resource-limited environments. Using a high-fidelity porcine model, this study demonstrates the neuroprotective efficacy of XeMBs in TBI for the first time.

Traumatic brain injury (TBI) is the leading cause of mortality and morbidity in children and young adults. The neurologic sequelae of TBI are affected not only by the type and severity of injury (primary injury) but also by a complex secondary pathophysiological cascade (secondary injury). For instance, TBI-induced axonal shear stretch results in the opening of voltage-gated calcium channels, triggering mitochondrial dysfunction, bioenergetic failure, and cell death pathways. There is a critical need to advance effective therapies that can be instituted quickly to prevent the progression of brain injury immediately after injury. At present, the standard of care is supportive in nature, focused on managing symptoms rather than treating the disease. Surgical intervention is instituted only in the case of severe brain injuries, i.e., life threatening hemorrhage or brain herniation.

In this report, we propose the utilization of microbubbles encapsulating the noble gas xenon to treat TBI in the immediate post-injury period. Xenon is an anesthetic gas with demonstrated neuroprotective effect in small animal models of TBI.

Neuroprotection by xenon has also been confirmed using a blast injury model of in vitro TBI with reduced injury as assessed by propidium iodide staining. In a mouse model of controlled cortical impact, inhaled xenon administration at 75%¹⁶ and 30-50% of the total gas composition were shown to diminish injury extent and improve neurological outcomes. Histologic analyses demonstrated reduced astrogliosis and microglia damage in the hypothalamus and amygdala, respectively. Additionally, there was an attenuation of hippocampal neuronal loss. Furthermore, while not in TBI, a randomized clinical trial demonstrated that inhaled xenon decreased cerebral white matter damage, measured by magnetic resonance imaging (MRI), in 110 comatose survivors of out-of-hospital cardiac arrest.

One reported mechanism of xenon-mediated neuroprotection is the antagonism of N-methyl-d-aspartate receptor (NMDA) subtype of glutamate receptors. As a potential neuroprotectant, xenon is hemodynamically safe, permeable to the blood brain barrier, and has a wide safety margin in vivo. Unfortunately, the extremely high cost and inconvenience of xenon inhalational delivery limits its widespread study and use clinically. Likely, the xenon inhalation delivery method achieves low xenon concentration in the brain, limiting its neuroprotective efficacy without high dose concentrations. In addition, systemic delivery of high doses of xenon increases the likelihood of unwanted side effects such as vomiting and nausea. It is important to recognize that, to our knowledge, the neuroprotective effect shown in the rodent TBI studies has never been shown in large animal TBI models nor in humans. Oftentimes, the therapeutic efficacy of novel drugs as determined from small animal models is not translatable to large animals or humans due to the marked differences in body habitus and drug pharmacokinetics.

To this end, our group has been developing a novel delivery technique that employs ultrasound-mediated release of xenon from microbubbles (MBs) into the brain via a localized application of a high acoustic impulse to the common carotid arteries following intravenous injection of xenon MBs (XeMBs). The over-arching goal is therefore to overcome current limitations in xenon delivery by devising a novel, clinically translatable ultrasound-guided delivery method that improves neurologic outcomes following TBI in a high-fidelity large animal model of focal TBI.

Methods

Formulation and Imaging of XeMBs

XeMBs were formulated as described previously. Briefly, 10 mL of a 10 mg/mL solution of DBPC (dibehenoylphosphatidylcholine) and DSPE-PEG5000 (in a 9:1 molar ratio) was purged with xenon gas for 10 min, followed by probe-sonication (Branson Digital Sonifier 250, 70% power) to create MBs. MBs were then size separated by centrifuging at 300 g for 5 min. For making control MBs, perfluorobutane was used instead of xenon, and a starting solution of 3 mg/mL lipids were sued. MBs were stored at 4° Celsius in a glass vial with the headspace purged with xenon or perfluorobutane, as applicable. MBs were used within 16-24 hours of preparation.

Neuroprotective Efficacy of XeMBs in Porcine Model of TBI

The study was performed under an approved Institutional Animal Care and Use Committee (IACUC) protocol. A well-established focal controlled cortical impact porcine model of TBI (1-month old, weighing approximately 10 kg) was utilized to assess the therapeutic efficacy of Perfluorobutane (PFB) MBs versus XeMBs. In brief, the right coronal suture was exposed, and a craniectomy performed over the rostral gyrus allowing a 1 cm margin around the indenter tip of the cortical impact device described previously. The exposed dura was opened in a stellate fashion to reveal the cortical surface, and the device stabilized against the skull with screws. The spring-loaded tip rapidly (4 ms) indented 0.63 cm of the cortical rostral gyms.

Day 1 and Day 5 MRIs were performed and conventional T1- and T2-weighted sequences analyzed to assess for changes in injury volume size in the two experimental groups (n=6 total, 3 in each group). Day 5 MRI is obtained to validate the therapeutic efficacy of XeMBs into the subacute period. The experimental design consisted of intravenous PFB MB or XeMB administration at 1, 3, and 24 hours. after injury using the following protocol per treatment session: continuous infusion at 0.2 mL/min for 1.5 min, 0.4 mL/min for 0.5 min, and 0.6 mL/min for 6 min, for a total of 8 minutes of infusion. Injected microbubble concentrations for both Xenon and PFB were adjusted to similar levels via necessary dilutions to a final number of approximately 10⁸-2×10⁸ per mL. Note that the dosing has been adjusted for pigs based on a previously performed preclinical study using rodents. A hand-held portable ultrasound device and probe (Lumify, Philips Healthcare) with a mechanical index of 1.0 was used to release the xenon gas from MBs at the level of the carotid artery, in longitudinal axis at frame rate between 18-20 frames per second and with frequency range of 5-10 MHz.

MRI Imaging

Day 1 and 5 MRIs were obtained on a Siemens 3T TRIO MRI research magnet (Siemens, Munich, Germany) using a standard knee coil. Anatomical images (T1-weighted, T2-weighted, and diffusion-weighted) were acquired. T1- and T2-weighted sequences were loaded into pMRI (www.parametricmri.com) software. Following manual delineation of edema (T2 hyperintense) and core (T2 hypointense) borders on each slice, a three-dimensional volume of injury was calculated and recorded using the volume module of pMRI. All tracings were performed by the consensus of two blinded reviewers, one of whom was a board-certified radiologist.

Histopathology

Anesthetized animals were sacrificed, and the brain was removed whole. The brains were then cut into standardized 5 mm coronal sections and fixed in 10% neutral buffered formalin. Following a fixation period of at least two weeks, whole brain slices were processed, paraffin embedded, cut into 5 μm sections, and stained with hematoxylin and eosin (H&E).

Histopathology studies were performed by an experienced board-certified neuropathologist. The reviewer was blinded. H&E-stained histologic sections of both hemispheres were examined to assess for the location, contents, and extent of the lesion. Perivascular inflammation was scored as follows: 0=no to rare inflammatory cells; 1=a few inflammatory cells; 2=cuff of inflammatory cells 1 cell layer thick; 3=cuff of inflammatory cells >1 cell layer thick. Vascular changes including endothelial proliferation were also examined and scored as follows: 0=no vascular changes; 1=reactive endothelial cells; 2=endothelial proliferation (>1 cell layer thick). Perivascular inflammation and vascular changes were scored in the ipsilateral cortex surrounding the lesion and in the contralateral hemisphere.

Statistical Analysis

An unpaired student's t-test was used to compare outcomes across groups. Statistical significance was defined as p<0.05.

Results

We have previously formulated microbubbles encapsulating pure xenon gas and tested its stability and echogenicity. We found that long-chain phospholipids like DBPC, which is known to form a rigid, coherent, gel phase below its melting temperature (74° C.)³⁴ are especially suited to produce stable MBs. These MBs were storage-stable and echogenic under clinical ultrasound in tissue-mimicking phantoms, making them the first echogenic pure noble gas microbubble theranostic agent. The amount of xenon in the MB suspension, estimated via gas chromatography-mass spectrometry (GC-MS), is 4.5 μmol/mL (˜10⁸ XeMBs/mL).

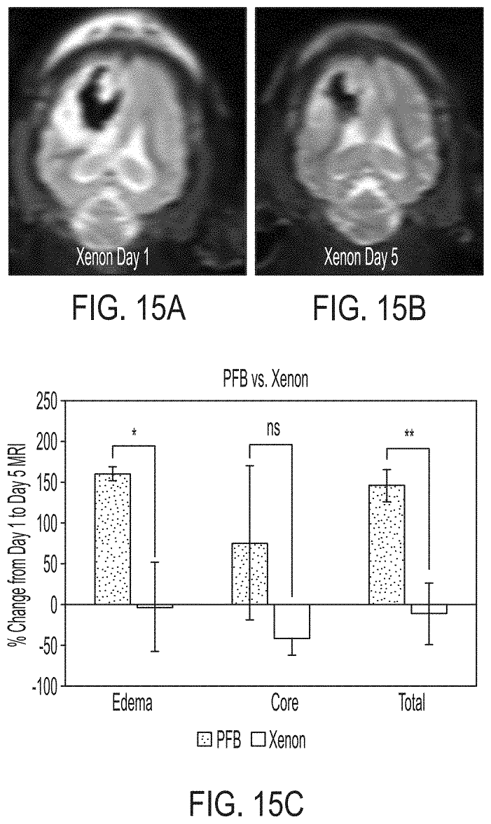

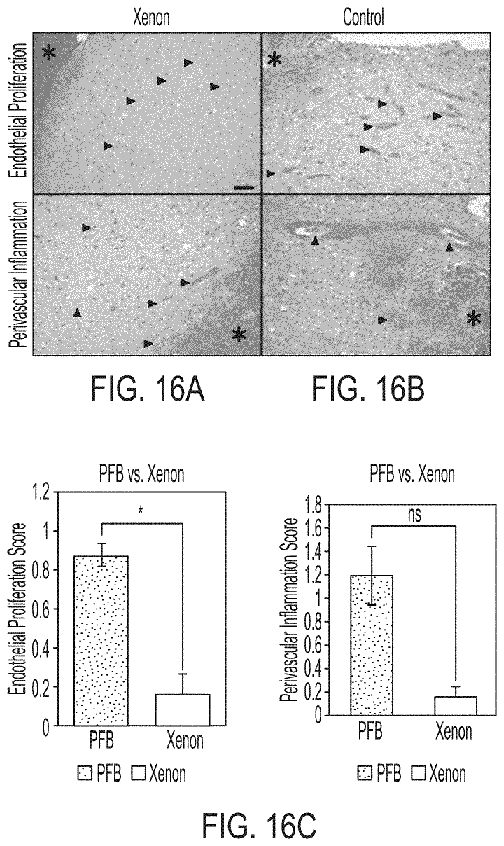

Neuroprotective efficacy of XeMBs in the porcine model of TBI was confirmed on brain magnetic resonance imaging (MRI) (FIG. 15) and neuropathology (FIG. 16). On MRI, significant increase in injury edema (T2 hyperintense) (p=0.03) and total volume (cm³) (p=0.01) in the control group from Day 1 to Day 5 MRI was demonstrated, with increased perilesional high signal on T2-weighted sequence in comparison to the markedly decreased perilesional edema in the xenon-treated group. Moreover, an increase in size of the core/hemorrhage (T2 hypointense) was noted in the control group. However, this finding was not statistically significant. Histological evaluation of the porcine brains demonstrated statistically significant decreased reactive vascular changes (p=0.002) in the xenon-treated group compared to the control group. Additionally, perivascular inflammation (including neutrophils, lymphocytes, and macrophages) adjacent to the site of injury was lower in animals treated with XeMBs in comparison to untreated, control animals. However, this was not a statistically significant finding.

Discussion

This report proposes a new clinically translatable, cost-effective, and convenient therapy that can be used in the acute TBI setting. Ultrasound-guided xenon delivery to the brain in the acute post-TBI period drastically reduced injury edema and volume on MRI as well as perilesional reactive endothelial changes and perivascular inflammation on histology. The shell components used for XeMBs are similar to that of commercially used, Federal Drug Administration (FDA) approved, ultrasound contrast MBs. Additionally, only requiring a portable, hand-held ultrasound system and probe to urgently institute XeMB therapy. Acoustically triggered release of xenon from its carrier microbubble at the level of the carotid artery enables targeted cerebral delivery of xenon. Our finding provides a paradigm shift from the current standard of care, wherein no effective therapy exists for treatment of TBI in the acute setting.

While xenon gas inhalation therapy has been tried in animal models and humans, the approach typically used for administering xenon, is expensive, inconvenient, and fraught with systemic side effects. Therapeutic delivery to the brain is significantly low, with much of the gas cleared via the lungs. Commercially available MBs used as ultrasound contrast agents for diagnostic imaging have an inert gas in the core (e.g., perfluorocarbons like PFB or Perfluoropropane, sulfur hexafluoride (SF₆), etc), encapsulated by a phospholipid monolayer. The ultrasound settings used are within the safety limitations of clinical use. A recent publication has demonstrated that targeted release of xenon from xenon encapsulated liposomes can achieve neuroprotection, but data on large animal models, particularly a TBI model, are lacking. To this end, this study provides the first evidence toward the promising translational utility of XeMBs for neuroprotection in TBI.

The reported results suggest mechanisms with which xenon affects endothelial cell proliferation and perivascular inflammation in the acute post-TBI period. Potential mechanisms of xenon-mediated neuroprotection include N-methyl-d-aspartate receptor (NMDA) subtype of glutamate receptor antagonism, potent activator of the two-pore domain K⁺ channel, shown to play an important role in neuroprotection in neuroprotection, upregulation of the pro-survival proteins Bcl-2 and brain-derived neurotrophic factor induction of the expression of hypoxia inducible factor 1α (HIF 1a) and its downstream effectors erythropoietin (EPO) and vascular endothelial growth factor (VEGF), and induction of trophic factors including brain derived neurotrophic factor.

Conclusion

Our data suggests that xenon microbubbles can drastically reduce the injury extent as assessed using magnetic resonance imaging. This therapy can transform the way in which acute TBI is treated and can be broadly adopted. The data presented provides the first proof-of-concept evidence toward the use of xenon microbubbles for treatment of acute TBI.

High Yield Production of Phospholipid Stabilized ArMBs with Prolonged in Vivo Echogenicity.

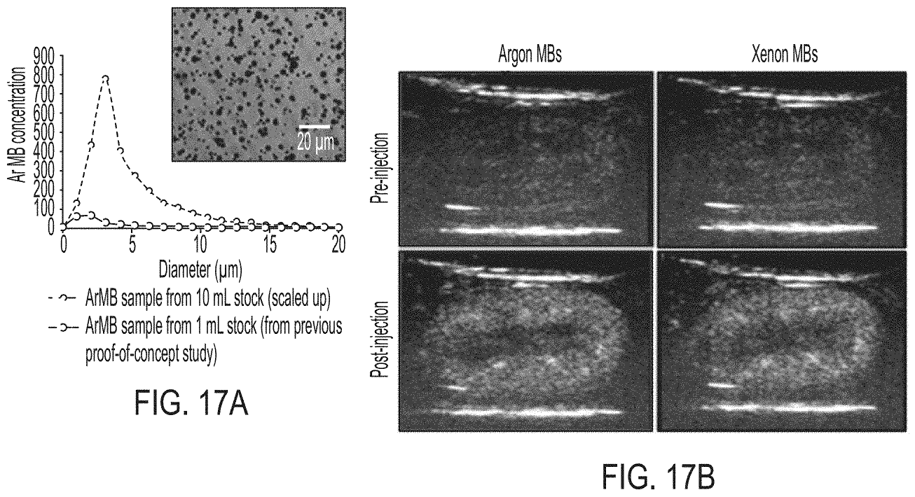

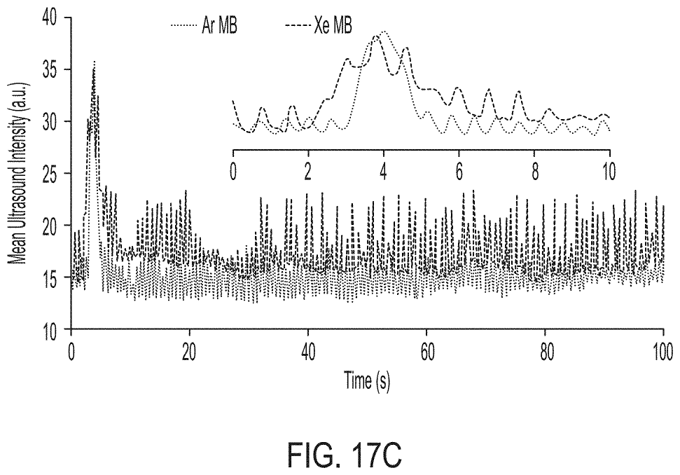

An objective was development of 1-10 μm echogenic microbubbles (MBs) encapsulating pure Ar gas as a cheap, safe, and more efficient alternative to on inhalation/bulk exposure Ar delivery, while being a markedly cheaper and widely available alternative to possible Xe-based therapy. We have previously prepared stable MBs with pure Xe or Ar cores in small amounts (<1 mL) as proof-of-concept. This was done via saturating with xenon or argon a 1 mL phospholipid solution of dibehenoylphophatidylcholine (DBPC) and DSPE-PEG5000 in a 9:1 molar ratio, followed by sonication with a ⅛″ probe at 10% power for 20 s (Branson Sonifier 250). The final bubble stocks were partially stable for several days. In our previous work with Xe MBs, to improve bubble stability, we had shown that scaling up the process by using a starting lipid stock of 10 mL, using a broader sonication tip (½″), and concentrating the final bubble yield down to the same volume helped keep bubbles stable. We had the objective to show that Ar MB production can be scaled up as well. Ar MBs were thus prepared with a starting stock of 10 mL lipids and stored in <1 mL volume of bubbles produced a much higher bubble yield than the previous results (FIG. 17A), paving the way for a straightforward method to formulate large quantities of stable Ar MBs.

Ar MBs can be echogenic in vivo, and we compared their echogenicity to xenon and perfluorocarbon MBs. To this end, 30 uL of equal concentrations (5×10⁸ per mL) of either ArMBs or XeMBs were injected retro-orbitally into a male C57BL/6J mouse under isoflurane anesthesia (protocol approved by IACUC). FIGS. 17B-17C shows pre- and post-injection contrast images of the kidney under ultrasound (Visualsonics VevoLazr, 18 MHz, 10% power). While the signal lasts momentarily after injection, ArMB signal is essentially equivalent to that of XeMBs, and will be persistently visible during a continuous infusion of ArMBs instead of a bolus during future therapy. This is the first reported instance of microbubbles made of pure argon showing discernible echogenicity in vivo, paving the way for image-guided, localized gas delivery for theranostic applications.

### EMBODIMENTS

The following embodiments are exemplary only and do not serve to limit the scope of the present disclosure or of the appended claims.

Embodiment 1. A microbubble composition, comprising: a plurality of microbubbles, a microbubble comprising a noble gas and/or a perfluorocarbon encapsulated within a shell that comprises one or more of a lipid, a protein, or a polymer, and a microbubble optionally defining a cross-sectional dimension in the range of from about 0.5 to about 20 micrometers.

The shell of a microbubble according to the present disclosure can have a thickness in the range of, e.g., from about 2 to about 10 nm, or from about 3 to about 8 nm, or even in the range of from about 3 to about 4 nm. The foregoing values are illustrative only and are not limiting. A phospholipid monolayer can have a thickness in the range of from about 2 to about 5 nm, or even from about 3 to about 4 nm.

The shell of a microbubble can have a stiffness in the range of from about 2 to about 3 N/m, e.g., from about 2 to about 3 N/m, from about 2.1 to about 2.9 N/m, from about 2.3 to about 2.8 N/m, from about 2.4 to about 2.7 N/m, or from about 2.5 to about 2.6 N/m. The foregoing values are illustrative only and are not limiting.

Without being bound to any particular theory, a MB composition that includes noble gas can include from about 100 to about 300 μL per 10⁸ MBs. As an example, for xenon-containing MBs, the composition can include about 4.5 μmol Xe/mL of MB solution injected. The amount of noble gas (and/or perfluorocarbon) administered to a patient can depend, of course, on the patient's condition and/or characteristics. As an example, one might provide 3 infusions of 3.5 mL of 10⁸ MB/mL each after a post-injury period to a porcine subject.

A microbubble can define a cross-sectional dimension in the range of from about 0.5 to about 20 micrometers, or from about 1 to about 19 micrometers, or from about 2 to about 18 micrometers, or from about 3 to about 17 micrometers, or from about 4 to about 16 micrometers, or from about 5 to about 15 micrometers, or from about 6 to about 14 micrometers, or from about 7 to about 13 micrometers, or from about 8 to about 12 micrometers, or from about 9 to about 11 micrometers, and all combined and/or subranges.

Embodiment 2. The microbubble composition of Embodiment 1, wherein the lipid comprises a phospholipid.

Embodiment 3. The microbubble composition of any one of Embodiments 1-2, wherein the polymer is a lipopolymer.

Embodiment 4. The microbubble composition of any one of Embodiments 1-3, wherein the shell comprises a phospholipid and/or a lipopolymer. Example phospholipids include, e.g., phosphatidic acid (phosphatidate) (PA), phosphatidylethanolamine (cephalin) (PE), phosphatidylcholine (lecithin) (PC), phosphatidylserine (PS), phosphatidylinositol (PI), phosphatidylinositol phosphate (PIP), phosphatidylinositol bisphosphate (PIP2), phosphatidylinositol trisphosphate (PIP3), ceramide phosphorylcholine (sphingomyelin) (SPH), ceramide phosphorylethanolamine (sphingomyelin) (Cer-PE), ceramide phosphoryllipid. A lipopolymer can include a lipid (fatty acid or steroid) moiety.

Embodiment 5. The microbubble composition of Embodiment 4, wherein the phospholipid comprises 1,2-dipalmitoyl-sn-glycero-3-phosphocholine (DPPC) (C16:0) or 1,2-dibehenoyl-sn-glycero-3-phosphocholine (DSPC) (C22:0). A shell can comprise, e.g., DSPC, DBPC, DPPC, DSPE-PEG2000, DSPE-PEG5000, DSPE-PEG3400, PEG40-stearate, and the like. A shell (in particular a shell used with an MB that contains a perfluorocarbon therein) can also comprise a lyso-PC (e.g., a lysophosphatidylcholine) and/or egg-PC (L-α-phosphatidylcholine).

Embodiment 6. The microbubble composition of any one of Embodiments 3-5, wherein the lipopolymer comprises polyethylene glycol.

Embodiment 7. The microbubble composition of any one of Embodiments 1-6, wherein a microbubble defines a cross-sectional dimension in the range of from about 1 to about 10 micrometers.

Embodiment 8. The microbubble composition of any one of Embodiments 1-7, wherein the noble gas comprises helium, argon, or xenon.

Embodiment 9. The microbubble composition of Embodiment 8, wherein the noble gas comprises xenon.

The perfluorocarbon can be, e.g., a perfluoroalkane, such as perfluoromethane, perfluoroethane, perfluoropropane, perfluoropentane, perfluorobutane, perfluoropentane, perfluorohexane, perfluoroheptane, perfluorooctane, and the like. The perfluorocarbon gas can be a linear chain, but can also be branched, cyclic, or even polycyclic. The perfluorocarbon can be one in which all C—H bonds are replaced with C—F bonds.

As described elsewhere herein, a MB can comprises therein a noble gas, a perfluorocarbon, or both. The mass ratio of the noble gas to the perflurorocarbon (within a given MB) can be from about 1000:1 to about 1:1000, or from about 500:1 to about 1:500, or from about 250:1 to about 1:250, or 100:1 to about 1:100, or from 50:1 to about 1:50, or from about 25:1 to about 1:25, or from about 10:1 to about 1:10, or from 5:1 to about 1:5, or even from about 2:1 to about 1:2, and all intermediate values.

Embodiment 10. The microbubble composition of Embodiment 8, wherein the shell comprises one or more targeting groups configured to bind to one or more complementary targets. Such a complementary target can be, e.g., a surface marker, an antigen,

Embodiment 11. The microbubble composition of Embodiment 10, wherein the targeting group comprises an antibody, a peptide, or an aptamer. As one example, one can use an RGD peptide to target integrins on general endothelial cells for MB adhesion.

Embodiment 12. The microbubble composition of Embodiment 8, further comprising a carrier fluid in which the plurality of microbubbles is disposed.

Embodiment 13. The microbubble composition of Embodiment 12, wherein the carrier fluid is biocompatible. Carrier fluids can be, e.g., saline, plasma, blood, and the like. A carrier fluid can be aqueous.

Embodiment 14. The microbubble composition of any one of Embodiments 1-13, wherein the plurality of microbubbles are characterized as being monodisperse.

Embodiment 15. The microbubble composition of any one of Embodiments 1-14, wherein the plurality of microbubbles are characterized as being polydisperse.

Embodiment 16. A method, comprising: forming a composition according to any one of Embodiments 1-15. Microbubbles can be formed by, e.g., dissolving a film-forming material (e.g., a phospholipid, a lipopolymer, and the like) in a solvent, which solvent is then evaporated to form a film. The film can be resuspended in a solvent, and the noble gas and/or perfluorocarbon can be bubbled into the resuspended film so as to form the microbubbles.

Embodiment 17. The method of Embodiment 16, wherein the forming comprises agitating the noble gas and/or perfluorocarbon and shell-forming materials.

Embodiment 18. The method of Embodiment 16, wherein the forming comprises applying acoustic energy to an interface between the noble gas and/or perfluorocarbon and shell-forming materials.

Embodiment 19. A method, comprising administering a microbubble composition according to any one of Embodiments 1-15 to a subject, the composition optionally comprising echogenic phospholipid microbubbles.

Embodiment 20. The method of Embodiment 19, wherein the administration comprises intravenous administration.

Embodiment 21. The method of any one of Embodiments 19-20, wherein the subject has suffered or is suspected of suffering an injury, e.g., a brain injury. Such a brain injury can be characterized as a traumatic brain injury (TBI); an injury can also be a hypoxia ischemia reperfusion injury, e.g., a stroke induced by cardiac arrest or hypoxic ischemic encephalopathy in neonates. Accordingly, hypoxia/ischemia injuries are example injuries that may be addressed by the disclosed technology.

Embodiment 22. The method of Embodiment 21, further comprising rupturing at least some of the microbubbles.

Embodiment 23. The method of Embodiment 22, the method of Embodiment 22, wherein the rupturing is effected by application of ultrasound energy. This can be accomplished by applying a clinically-acceptable level of ultrasound.

Embodiment 24. The method of any one of Embodiments 22-23, wherein the microbubbles are ruptured while located in a vessel downstream from the brain. As an example (and without being bound to any particular theory), one can apply an ultrasound pulse along the carotid artery at the level of the neck upon intravenous injection of noble gas microbubbles (e.g., xenon-containing microbubbles), resulting in release of the noble gas from the microbubbles into the brain. (As described elsewhere herein, the microbubbles can also comprise a perfluorocarbon.) One can also apply the ultrasound pulse to a different blood vessel upstream of the brain.

Embodiment 25. The method of any one of Embodiments 19-24, further comprising identifying, with application of ultrasound energy, the location of the microbubble composition, e.g., imaging.

Without being bound to any particular theory or embodiment, the MI used in an imaging application be in the range of from about 0.05 to about 0.2, e.g., from about 0.08 to about 0.2 from about 0.1 to about 0.18, from about 0.12 to about 0.16, or from about 0.13 to about 0.15. The MI used for MB rupture can be, e.g., from about 0.8 to about 1.7, e.g., from about 0.8 to about 1.7, from about 0.9 to about 1.6, from about 1.0 to about 1.5, from about 1.1 to about 1.4, or even from about 1.2 to about 1.3.

In some clinical settings, the MI can be in the range of from about 0.05 to about 1.9 (depending on the application); the maximum MI on commercial ultrasound scanners can be 1.9 in some instances.

Without being bound to any particular theory or embodiment, rupture can be accomplished (depending on the frequency used) at from about 0.8 to 2.5 MPa peak negative pressures for rupture (corresponding to a frequency of from about 1 to about 10 MHz). As some examples, the peak pressure can be from about 0.8 to about 2.5 MPa, or from about 0.9 to about 2.4 MPa, or from about 1 to about 2.3 MPa, or from about 1.1 to about 2.2 MPa, or from about 1.3 to about 2.1 MPa, or from about 1.4 to about 2 MPa, or from about 1.5 to about 1.9 MPa, or from about 1.6 to about 1.8 MPa. Clinical frequencies can range from about 1 to about 10 MHz, e.g., from about 1 to about 5 MHz. Frequencies of less than 5 MHz are considered especially suitable.

Embodiment 26. A method, comprising: (a) identifying, with the application of energy, the location of a microbubble composition according to any one of Embodiments 1-15, the energy optionally being ultrasound, (b) controllably effecting rupture of microbubbles of a microbubble composition of any one of Embodiments 1-15, the rupture optionally being effected by application of ultrasound, or both (a) and (b).

Embodiment 27. The method of Embodiment 26, wherein the microbubbles are disposed within a subject.

### REFERENCES

K. F. Smit, N. C. Weber, M. W. Hollmann, B. Preckel, Noble gases as cardioprotectants—translatability and mechanism, Br. J. Pharmacol. 172 (2015) 2062-2073. doi:10.1111/bph.12994.
C.-S. Zhao, H. Li, Z. Wang, G. Chen, Potential application value of xenon in stroke treatment, Med. Gas Res. 8 (2018) 116-120. doi:10.4103/2045-9912.241077.
E. Esencan, S. Yuksel, Y. B. Tosun, A. Robinot, I. Solaroglu, J. H. Zhang, XENON in medical area: emphasis on neuroprotection in hypoxia and anesthesia, Med. Gas Res. 3 (2013) 4. doi:10.1186/2045-9912-3-4.
W. Liu, Y. Liu, H. Chen, K. Liu, H. Tao, X. Sun, Xenon preconditioning: molecular mechanisms and biological effects, Med. Gas Res. 3 (2013) 3. doi:10.1186/2045-9912-3-3.
B. Preckel, N. C. Weber, R. D. Sanders, M. Maze, W. Schlack, Molecular Mechanisms Transducing the Anesthetic, Analgesic, and Organ-protective Actions of Xenon, Anesthesiol. J. Am. Soc. Anesthesiol. 105 (2006) 187-197.
N. Jawad, M. Rizvi, J. Gu, O. Adeyi, G. Tao, M. Maze, D. Ma, Neuroprotection (and lack of neuroprotection) afforded by a series of noble gases in an in vitro model of neuronal injury, Neurosci. Lett. 460 (2009) 232-236. doi:10.1016/j.neulet.2009.05.069.
P. D. Loetscher, J. Rossaint, R. Rossaint, J. Weis, M. Fries, A. Fahlenkamp, Y.-M. Ryang, O. Grottke, M. Coburn, Argon: Neuroprotection in in vitro models of cerebral ischemia and traumatic brain injury, Crit. Care. 13 (2009) R206. doi:10.1186/cc8214.
A. Brücken, A. Cizen, C. Fera, A. Meinhardt, J. Weis, K. Nolte, R. Rossaint, T. Pufe, G. Marx, M. Fries, Argon reduces neurohistopathological damage and preserves functional recovery after cardiac arrest in rats, BJA Br. J. Anaesth. 110 (2013) i106—i112. doi:10.1093/bja/aes509.
M. Coburn, M. Maze, N. Franks, The neuroprotective effects of xenon and helium in an in vitro model of traumatic brain injury*, Crit. Care Med. 36 (2008) 588-595. doi:10.1097/01.CCM.0B013E3181611F8A6.
H. N. David, B. Haelewyn, L. Chazalviel, M. Lecocq, M. Degoulet, J.-J. Risso, J. H. Abraini, Post-Ischemic Helium Provides Neuroprotection in Rats Subjected to Middle Cerebral Artery Occlusion-Induced Ischemia by Producing Hypothermia, J. Cereb. Blood Flow Metab. 29 (2009) 1159-1165. doi:10.1038/jcbfm.2009.40.
M. Rizvi, N. Jawad, Y. Li, M. P. Vizcaychipi, M. Maze, D. Ma, Effect of noble gases on oxygen and glucose deprived injury in human tubular kidney cells, Exp. Biol. Med. 235 (2010) 886-891. doi:10.1258/ebm.2010.009366.
S. Dähn, P. Schwalbach, S. Maksan, F. Wöhleke, A. Benner, C. Kuntz, Influence of different gases used for laparoscopy (helium, carbon dioxide, room air, and xenon) on tumor volume, histomorphology, and leukocyte—tumor—endothelium interaction in intravital microscopy, Surg. Endosc. Interv. Tech. 19 (2005) 65-70. doi:10.1007/s00464-003-9298-z.
Xenon and Cooling Therapy in Babies at High Risk of Brain Injury Following Poor Condition at Birth—Full Text View—ClinicalTrials.gov, (n.d.). https://clinicaltrials.govict2/show/NCT02071394 (accessed Aug. 3, 2019).
Neuroprotective Effects of Hypothermia Combined With Inhaled Xenon Following Perinatal Asphyxia—Full Text View—ClinicalTrials.gov, (n.d.). https://clinicaltrials.govict2/show/NCT00934700 (accessed Aug. 3, 2019).
Britton George L., Kim Hyunggun, Kee Patrick H., Aronowski Jaroslaw, Holland Christy K., McPherson David D., Huang Shao-Ling, In Vivo Therapeutic Gas Delivery for Neuroprotection With Echogenic Liposomes, Circulation. 122 (2010) 1578-1587. doi:10.1161/CIRCULATIONAHA.109.879338.
Y.-F. Miao, T. Peng, M. R. Moody, M. E. Klegerman, J. Aronowski, J. Grotta, D. D. McPherson, H. Kim, S.-L. Huang, Delivery of xenon-containing echogenic liposomes inhibits early brain injury following subarachnoid hemorrhage, Sci. Rep. 8 (2018) 450. doi:10.1038/s41598-017-18914-6.
M. P. Dandekar, T. Peng, D. D. McPherson, J. Quevedo, J. C. Soares, S.-L. Huang, Intravenous infusion of xenon-containing liposomes generates rapid antidepressant-like effects, Prog. Neuropsychopharmacol. Biol. Psychiatry. 86 (2018) 140-149. doi:10.1016/j.pnpbp.2018.03.011.
S. M. Fix, M. A. Borden, P. A. Dayton, Therapeutic gas delivery via microbubbles and liposomes, J. Controlled Release. 209 (2015) 139-149. doi:10.1016/j.jconre1.2015.04.027.
S. R. Sirsi, M. A. Borden, Microbubble compositions, properties and biomedical applications, Bubble Sci. Eng. Technol. 1 (2009) 3-17. doi:10.1179/175889709X446507.
L. Hoff, P. C. Sontum, J. M. Hovem, Oscillations of polymeric microbubbles: Effect of the encapsulating shell, J. Acoust. Soc. Am. 107 (2000) 2272-2280. doi:10.1121/1.428557.
S. Hernot, A. L. Klibanov, Microbubbles in ultrasound-triggered drug and gene delivery, Adv. Drug Deliv. Rev. 60 (2008) 1153-1166. doi:10.1016/j.addr.2008.03.005.
E. J. Swanson, V. Mohan, J. Kheir, M. A. Borden, Phospholipid-Stabilized Microbubble Foam for Injectable Oxygen Delivery, Langmuir. 26 (2010) 15726-15729. doi:10.1021/1a1029432.
J. N. Kheir, L. A. Scharp, M. A. Borden, E. J. Swanson, A. Loxley, J. H. Reese, K. J. Black, L. A. Velazquez, L. M. Thomson, B. K. Walsh, K. E. Mullen, D. A. Graham, M. W. Lawlor, C. Brugnara, D. C. Bell, F. X. McGowan, Oxygen Gas—Filled Microparticles Provide Intravenous Oxygen Delivery, Sci. Transl. Med. 4 (2012) 140ra88-140ra88. doi:10.1126/scitranslmed.3003679.
J. A. Feshitan, N. D. Legband, M. A. Borden, B. S. Terry, Systemic oxygen delivery by peritoneal perfusion of oxygen microbubbles, Biomaterials. 35 (2014) 2600-2606. doi:10.1016/j.biomaterials.2013.12.070.
J. Tong, J. Ding, X. Shen, L. Chen, Y. Bian, G. Ma, Y. Yao, F. Yang, Mesenchymal Stem Cell Transplantation Enhancement in Myocardial Infarction Rat Model under Ultrasound Combined with Nitric Oxide Microbubbles, PLOS ONE. 8 (2013) e80186. doi:10.1371/journal.pone.0080186.
H. Shekhar, A. Palaniappan, T. Peng, M. Lafond, M. R. Moody, K. J. Haworth, S. Huang, D. D. McPherson, C. K. Holland, Characterization and Imaging of Lipid-Shelled Microbubbles for Ultrasound-Triggered Release of Xenon, Neurotherapeutics. (2019). doi:10.1007/s13311-019-00733-4.
L. H. Gevantman, SOLUBILITY OF SELECTED GASES IN WATER, (n.d.) 4.
J. J. Kwan, M. Kaya, M. A. Borden, P. A. Dayton, Theranostic Oxygen Delivery Using Ultrasound and Microbubbles, Theranostics. 2 (2012) 1174-1184. doi:10.7150/thno.4410.
R. D. Booker, A. K. Sum, Biophysical changes induced by xenon on phospholipid bilayers, Biochim. Biophys. Acta BBA—Biomembr. 1828 (2013) 1347-1356. doi:10.1016/j.bbamem.2013.01.016.
S. Garg, A. A. Thomas, M. A. Borden, The effect of lipid monolayer in-plane rigidity on in vivo microbubble circulation persistence, Biomaterials. 34 (2013) 6862-6870. doi:10.1016/j.biomaterials.2013.05.053.
E. Stride, M. Edirisinghe, Novel microbubble preparation technologies, Soft Matter. 4 (2008) 2350-2359. doi:10.1039/B809517P.
M. A. Parrales, J. M. Fernandez, M. Perez-Saborid, J. A. Kopechek, T. M. Porter, Acoustic characterization of monodisperse lipid-coated microbubbles: Relationship between size and shell viscoelastic properties, J. Acoust. Soc. Am. 136 (2014) 1077-1084. doi:10.1121/1.4890643.
T. Segers, P. Kruizinga, M. P. Kok, G. Lajoinie, N. de Jong, M. Versluis, Monodisperse Versus Polydisperse Ultrasound Contrast Agents: Non-Linear Response, Sensitivity, and Deep Tissue Imaging Potential, Ultrasound Med. Biol. 44 (2018) 1482-1492. doi:10.1016/j.ultrasmedbio.2018.03.019.
J. A. Feshitan, C. C. Chen, J. J. Kwan, M. A. Borden, Microbubble size isolation by differential centrifugation, J. Colloid Interface Sci. 329 (2009) 316-324. doi:10.1016/j.jcis.2008.09.066.
J. N. Israelachvili, Intermolecular and Surface Forces, Academic Press, 2015.
T. Brotin, J.-P. Dutasta, Cryptophanes and Their Complexes—Present and Future, Chem. Rev. 109 (2009) 88-130. doi:10.1021/cr0680437.
S. D. Zemerov, B. W. Roose, M. L. Greenberg, Y. Wang, I. J. Dmochowski, Cryptophane Nanoscale Assemblies Expand 129Xe NMR Biosensing, Anal. Chem. 90 (2018) 7730-7738. doi:10.1021/acs.analchem.8b01630.
P. A. Hill, Q. Wei, R. G. Eckenhoff, I. J. Dmochowski, Thermodynamics of Xenon Binding to Cryptophane in Water and Human Plasma, J. Am. Chem. Soc. 129 (2007) 9262-9263. doi:10.1021/ja072965p.
M. E. Klegerman, M. R. Moody, J. R. Hurling, T. Peng, S.-L. Huang, D. D.
McPherson, Gas chromatography/mass spectrometry measurement of xenon in gas-loaded liposomes for neuroprotective applications, Rapid Commun. Mass Spectrom. 31 (2017) 1-8. doi:10.1002/rcm.7749.
T. Seto, H. Isogai, M. Ozaki, S. Nosaka, Noble Gas Binding to Human Serum Albumin Using Docking Simulation: Nonimmobilizers and Anesthetics Bind to Different Sites, Anesth. Analg. 107 (2008) 1223. doi:10.1213/ane.0b013e31817f1317.
K. Glasmästar, C. Larsson, F. Höök, B. Kasemo, Protein Adsorption on Supported Phospholipid Bilayers, J. Colloid Interface Sci. 246 (2002) 40-47. doi:10.1006/jcis.2001.8060.
D. Harries, S. May, A. Ben-Shaul, Adsorption of charged macromolecules on mixed fluid membranes, Colloids Surf. Physicochem. Eng. Asp. 208 (2002) 41-50. doi:10.1016/S0927-7757(02)00100-0.
M. A. Borden, C. F. Caskey, E. Little, R. J. Gillies, K. W. Ferrara, DNA and Polylysine Adsorption and Multilayer Construction onto Cationic Lipid-Coated Microbubbles, Langmuir. 23 (2007) 9401-9408. doi:10.1021/1a7009034.
M. A. Borden, M. L. Longo, Oxygen Permeability of Fully Condensed Lipid Monolayers, J. Phys. Chem. B. 108 (2004) 6009-6016. doi:10.1021/jp037815p.
J. Sun, M. Yin, S. Zhu, L. Liu, Y. Zhu, Z. Wang, R. X. Xu, S. Chang, Ultrasound-mediated destruction of oxygen and paclitaxel loaded lipid microbubbles for combination therapy in hypoxic ovarian cancer cells, Ultrason. Sonochem. 28 (2016) 319-326. doi:10.1016/j.ultsonch.2015.08.009.
Z. Chen, R. Chattaraj, K. W. Pulsipher, M. B. Karmacharya, D. A. Hammer, D. Lee, C. M. Sehgal, Photoacoustic and Ultrasound Dual-Mode Imaging via Functionalization of Recombinant Protein-Stabilized Microbubbles with Methylene Blue, ACS Appl. Bio Mater. (2019). doi:10.1021/acsabm.9b00545.
Apfel R E, Holland C K. Gauging the likelihood of cavitation from short-pulse, low-duty cycle diagnostic ultrasound. Ultrasound Med Biol Elsevier, 1991; 17:179-185.
Back S J, Edgar J C, Canning D A, Darge K. Contrast-enhanced voiding urosonography: in vitro evaluation of a second-generation ultrasound contrast agent for in vivo optimization. Pediatr Radiol 2015; 45:1496-1505.
Blum N T, Yildirim A, Chattaraj R, Goodwin A P. Nanoparticles Formed by Acoustic Destruction of Microbubbles and Their Utilization for Imaging and Effects on Therapy by High Intensity Focused Ultrasound. Theranostics 2017; 7:694-702.
Borden M A, Kruse D E, Caskey C F, Shukui Zhao, Dayton P A, Ferrara K W. Influence of lipid shell physicochemical properties on ultrasound-induced microbubble destruction. IEEE Trans Ultrason Ferroelectr Freq Control 2005; 52:1992-2002.
Borden M A, Longo M L. Dissolution Behavior of Lipid Monolayer-Coated, Air-Filled Microbubbles: Effect of Lipid Hydrophobic Chain Length. Langmuir American Chemical Society, 2002; 18:9225-9233.
Borden M A, Song K-H. Reverse engineering the ultrasound contrast agent. Adv Colloid Interface Sci 2018; 262:39-49.
Chattaraj R, Hwang M, Zemerov S D, Dmochowski I J, Hammer D A, Lee D, Sehgal C M. Ultrasound Responsive Noble Gas Microbubbles for Applications in Image-Guided Gas Delivery. Adv Healthc Mater 2020; 9:1901721.
Chatterjee D, Jain P, Sarkar K. Ultrasound-mediated destruction of contrast microbubbles used for medical imaging and drug delivery. Phys Fluids American Institute of Physics, 2005; 17:100603.
Chen C C, Sirsi S R, Homma S, Borden M A. Effect of Surface Architecture on In Vivo Ultrasound Contrast Persistence of Targeted Size-Selected Microbubbles. Ultrasound Med Biol 2012; 38:492-503.
Chen Z, Chattaraj R, Pulsipher K W, Karmacharya M B, Hammer D A, Lee D, Sehgal C M. Photoacoustic and Ultrasound Dual-Mode Imaging via Functionalization of Recombinant Protein-Stabilized Microbubbles with Methylene Blue. ACS Appl Bio Mater American Chemical Society, 2019a; 2:4020-4026.
Chen Z, Pulsipher K W, Chattaraj R, Hammer D A, Sehgal C M, Lee D. Engineering the Echogenic Properties of Microfluidic Microbubbles Using Mixtures of Recombinant Protein and Amphiphilic Copolymers. Langmuir 2019b; 35:10079-10086.
Chen Z, Sultan L R, Schultz S M, Cary T W, Sehgal C M. Brachial flow-mediated dilation by continuous monitoring of arterial cross-section with ultrasound imaging. Ultrasound SAGE Publications, 2019c; 27:241-251.
Cosgrove D, Lassau N. Imaging of perfusion using ultrasound. Eur J Nucl Med Mol Imaging 2010; 37:65-85.
Eatock B C, Nishi R Y, Johnston G W. Numerical studies of the spectrum of low-intensity ultrasound scattered by bubbles. J Acoust Soc Am Acoustical Society of America, 1985; 77:1692-1701.
Esencan E, Yuksel S, Tosun Y B, Robinot A, Solaroglu I, Zhang J H. XENON in medical area: emphasis on neuroprotection in hypoxia and anesthesia. Med Gas Res 2013; 3:4.
Feshitan J A, Legband N D, Borden M A, Terry B S. Systemic oxygen delivery by peritoneal perfusion of oxygen microbubbles. Biomaterials 2014; 35:2600-2606.
Fix S M, Borden M A, Dayton P A. Therapeutic gas delivery via microbubbles and liposomes. J Controlled Release 2015; 209:139-149.
Forsberg F, Shi W T, Merritt CRB, Dai Q, Solcova M, Goldberg B B. On the Usefulness of the Mechanical Index Displayed on Clinical Ultrasound Scanners for Predicting Contrast Microbubble Destruction. J Ultrasound Med 2005; 24:443-450.
Garg S, Thomas A A, Borden M A. The effect of lipid monolayer in-plane rigidity on in vivo microbubble circulation persistence. Biomaterials 2013; 34:6862-6870.
Guo X, Li Q, Zhang Z, Zhang D, Tu J. Investigation on the inertial cavitation threshold and shell properties of commercialized ultrasound contrast agent microbubbles. J Acoust Soc Am Acoustical Society of America, 2013; 134:1622-1631.
Hemot S, Klibanov A L. Microbubbles in ultrasound-triggered drug and gene delivery. Adv Drug Deliv Rev 2008; 60:1153-1166.
Hoff L, Sontum P C, Hovem J M. Oscillations of polymeric microbubbles: Effect of the encapsulating shell. J Acoust Soc Am 2000; 107:2272-2280.
Huynh E, Lovell J F, Helfield B L, Jeon M, Kim C, Goertz D E, Wilson B C, Zheng G. Porphyrin Shell Microbubbles with Intrinsic Ultrasound and Photoacoustic Properties. J Am Chem Soc American Chemical Society, 2012; 134:16464-16467.
Irani Y, Pype J L, Martin A R, Chong C F, Daniel L, Gaudart J, Ibrahim Z, Magalon G, Lemaire M, Hardwigsen J. Noble Gas (Argon and Xenon)-Saturated Cold Storage Solutions Reduce Ischemia-Reperfusion Injury in a Rat Model of Renal Transplantation. Nephron Extra 2011; 1:272-282.
Kamotani Y, Lee WMF, Arger P H, Cary T W, Sehgal C M. Multigated contrast-enhanced power Doppler to measure blood flow in mice tumors. Ultrasound Med Biol 2003; 29:977-984.
Kheir J N, Scharp L A, Borden M A, Swanson E J, Loxley A, Reese J H, Black K J, Velazquez L A, Thomson L M, Walsh B K, Mullen K E, Graham D A, Lawlor M W, Brugnara C, Bell D C, McGowan F X. Oxygen Gas—Filled Microparticles Provide Intravenous Oxygen Delivery. Sci Transl Med 2012; 4:140ra88-140ra88.
Kwan J J, Borden M A. Microbubble Dissolution in a Multigas Environment. Langmuir American Chemical Society, 2010; 26:6542-6548.
Kwan J J, Borden M A. Lipid monolayer collapse and microbubble stability. Adv Colloid Interface Sci 2012; 183-184:82-99.
Kwan J J, Kaya M, Borden M A, Dayton P A. Theranostic Oxygen Delivery Using Ultrasound and Microbubbles. Theranostics 2012; 2:1174-1184.
Leong-Poi H, Song J, Rim S-J, Christiansen J, Kaul S, Lindner J R. Influence of microbubble shell properties on ultrasound signal: Implications for low-power perfusion imaging. J Am Soc Echocardiogr 2002; 15:1269-1276.
Li Q, Lian C, Zhou R, Li T, Xiang X, Liu B. Pretreatment With Xenon Protected Immature Rabbit Heart From Ischaemia/Reperfusion Injury by Opening of the mitoKATP Channel. Heart Lung Circ 2013; 22:276-283.
Lucidarme O, Kono Y, Corbeil J, Choi S-H, Mattrey R F. Validation of ultrasound contrast destruction imaging for flow quantification. Ultrasound Med Biol 2003; 29:1697-1704.
Lum J S, Stobbe D M, Borden M A, Murray T W. Photoacoustic technique to measure temperature effects on microbubble viscoelastic properties. Appl Phys Lett American Institute of Physics, 2018; 112:111905.
Ma D, Hossain M, Pettet G K, Luo Y, Lim T, Akimov S, Sanders R D, Franks N P, Maze M. Xenon Preconditioning Reduces Brain Damage from Neonatal Asphyxia in Rats. J Cereb Blood Flow Metab 2006; 26:199-208. Marmottant P, van der Meer S, Emmer M, Versluis M, de Jong N, Hilgenfeldt S, Lohse D. A model for large amplitude oscillations of coated bubbles accounting for buckling and rupture. J Acoust Soc Am Acoustical Society of America, 2005; 118:3499-3505.
Miao Y-F, Peng T, Moody M R, Klegerman M E, Aronowski J, Grotta J, McPherson D D, Kim H, Huang S-L. Delivery of xenon-containing echogenic liposomes inhibits early brain injury following subarachnoid hemorrhage. Sci Rep 2018; 8:450.
Partovi S, Loebe M, Aschwanden M, Baldi T, Jager K A, Feinstein S B, Staub D. Contrast-Enhanced Ultrasound for Assessing Carotid Atherosclerotic Plaque Lesions. Am J Roentgenol American Roentgen Ray Society, 2012; 198:W13—W19.
Reusser T D, Song K-H, Ramirez D, Benninger R K, Papadopoulou V, Borden M A. Phospholipid Oxygen Microbubbles for Image-Guided Therapy. Nanotheranostics 2020; 4:83-90.
Sarkar K, Katiyar A, Jain P. Growth and Dissolution of an Encapsulated Contrast Microbubble: Effects of Encapsulation Permeability. Ultrasound Med Biol 2009; 35:1385-1396.
Segers T, Kruizinga P, Kok M P, Lajoinie G, de Jong N, Versluis M. Monodisperse Versus Polydisperse Ultrasound Contrast Agents: Non-Linear Response, Sensitivity, and Deep Tissue Imaging Potential. Ultrasound Med Biol 2018; 44:1482-1492.
Shekhar H, Palaniappan A, Peng T, Lafond M, Moody M R, Haworth K J, Huang S, McPherson D D, Holland C K. Characterization and Imaging of Lipid-Shelled Microbubbles for Ultrasound-Triggered Release of Xenon. Neurotherapeutics 2019 [cited 2019 Aug. 3]; Available from: https://doi.org/10.1007/s13311-019-00733-4
Sieno L D, Cubeddu R, Sportouche H, Savery D, Sekar SKV, Rosinski B, Farina A, Ferocino E, Lanka P, Taroni P, Taroni P, Pifferi A, Pifferi A, Mora A D. Solid heterogeneous phantoms for multimodal ultrasound and diffuse-optical imaging: an outcome of the SOLUS project for standardization. Clin Preclin Opt Diagn I I 2019 Pap 1107539 Optical Society of America, 2019 [cited 2021 Feb. 20]. p. 11075_39. Available from: https://www.osapublishing.org/abstract.cfm?uri=ECB0-2019-1107539
Sijl J, Overvelde M, Dollet B, Garbin V, de Jong N, Lohse D, Versluis M. “Compression-only” behavior: A second-order nonlinear response of ultrasound contrast agent microbubbles. J Acoust Soc Am Acoustical Society of America, 2011; 129:1729-1739.
Sirsi S R, Borden M A. Microbubble compositions, properties and biomedical applications. Bubble Sci Eng Technol 2009; 1:3-17.
Szabo T. Diagnostic Ultrasound Imaging: Inside Out—2nd Edition. 2013 [cited 2021 Apr. 19]. Available from: https://www.elsevier.com/books/diagnostic-ultrasound-imaging-inside-out/szabo/978-0-12-396487-8
van Rooij T, Luan Y, Renaud G, van der Steen AFW, Versluis M, de Jong N, Kooiman K. Non-linear Response and Viscoelastic Properties of Lipid-Coated Microbubbles: DSPC versus DPPC. Ultrasound Med Biol 2015; 41:1432-1445.
Voorneveld J, Engelhard S, Vos H J, Reijnen MMPJ, Gijsen F, Versluis M, Jebbink E G, Jong N de, Bosch J G. High-Frame-Rate Contrast-Enhanced Ultrasound for Velocimetry in the Human Abdominal Aorta. IEEE Trans Ultrason Ferroelectr Freq Control 2018; 65:2245-2254.
Wang S, Mauldin F W, Klibanov A L, Hossack J A. Shear Forces from Flow Are Responsible for a Distinct Statistical Signature of Adherent Microbubbles in Large Vessels. Mol Imaging SAGE Publications Inc, 2013; 12:7290.2013.00057.
Wilson S R, Burns P N. Microbubble-enhanced U S in Body Imaging: What Role? Radiology Radiological Society of North America, 2010; 257:24-39.
Wood AKW, Schultz S M, Lee W M-F, Bunte R M, Sehgal C M. Antivascular Ultrasound Therapy Extends Survival of Mice With Implanted Melanomas. Ultrasound Med Biol 2010; 36:853-857.
Zhang, Yi. Measuring acoustic attenuation of polymer materials using drop ball test. ERAU Scholarly Commons, 2013.
Heron, M., Sutton, P. D., Xu, J., Ventura, S. J., Strobino, D. M., and Guyer, B. (2010). Annual summary of vital statistics: 2007. Pediatrics 125, 4-15.
Chelly, H., Chaari, A., Daoud, E., Dammak, H., Medhioub, F., Mnif, J., Hamida, C. B., Bahloul, M., and Bouaziz, M. (2011). Diffuse axonal injury in patients with head injuries: an epidemiologic and prognosis study of 124 cases. J. Trauma 71, 838-846.
McIntosh, T. K., Smith, D. H., Meaney, D. F., Kotapka, M. J., Gennarelli, T. A., and Graham, D. I. (1996). Neuropathological sequelae of traumatic brain injury: relationship to neurochemical and biomechanical mechanisms. Lab. Invest. 74, 315-342.
Gennarelli, T. A. (1993). Mechanisms of brain injury. J. Emerg. Med. 11 Suppl 1, 5-11.
Gennarelli, T. A. (1997). The pathobiology of traumatic brain injury. Neuroscientist 3, 73-81.
McIntosh, Saatman, Raghupathi, Graham, Smith, Lee, and Trojanowski. (1998). The molecular and cellular sequelae of experimental traumatic brain injury: pathogenetic mechanisms. Neuropathol Appl Neurobiol 24, 251-267. Marion, D. W. (1999). Traumatic brain injury. Thieme Medical Publisher, New York, N. Y.
Loane, D. J., and Faden, A. I. (2010). Neuroprotection for traumatic brain injury: translational challenges and emerging therapeutic strategies. Trends Pharmacol. Sci. 31, 596-604.
Balan, I. S., Saladino, A. J., Aarabi, B., Castellani, R. J., Wade, C., Stein, D. M., Eisenberg, H. M., Chen, H. H., and Fiskum, G. (2013). Cellular alterations in human traumatic brain injury: changes in mitochondrial morphology reflect regional levels of injury severity. J. Neurotrauma 30, 367-381.
Glenn, T. C., Kelly, D. F., Boscardin, W. J., McArthur, D. L., Vespa, P., Oertel, M., Hovda, D. A., Bergsneider, M., Hillered, L., and Martin, N. A. (2003). Energy dysfunction as a predictor of outcome after moderate or severe head injury: indices of oxygen, glucose, and lactate metabolism. J. Cereb. Blood Flow Metab. 23, 1239-1250.
Lifshitz, J., Friberg, H., Neumar, R. W., Raghupathi, R., Welsh, F. A., Janmey, P., Saatman, K. E., Wieloch, T., Grady, M. S., and McIntosh, T. K. (2003). Structural and functional damage sustained by mitochondria after traumatic brain injury in the rat: evidence for differentially sensitive populations in the cortex and hippocampus. J. Cereb. Blood Flow Metab. 23, 219-231.
Marcoux, J., McArthur, D. A., Miller, C., Glenn, T. C., Villablanca, P., Martin, N. A., Hovda, D. A., Alger, J. R., and Vespa, P. M. (2008). Persistent metabolic crisis as measured by elevated cerebral microdialysis lactate-pyruvate ratio predicts chronic frontal lobe brain atrophy after traumatic brain injury. Crit. Care Med. 36, 2871-2877.
Ragan, D. K., McKinstry, R., Benzinger, T., Leonard, J. R., and Pineda, J. A. (2013). Alterations in cerebral oxygen metabolism after traumatic brain injury in children. J. Cereb. Blood Flow Metab. 33, 48-52.
Xu, Y., McArthur, D. L., Alger, J. R., Etchepare, M., Hovda, D. A., Glenn, T. C., Huang, S., Dinov, I., and Vespa, P. M. (2010). Early nonischemic oxidative metabolic dysfunction leads to chronic brain atrophy in traumatic brain injury. J. Cereb. Blood Flow Metab. 30, 883-894.
Campos-Pires, R., Koziakova, M., Yonis, A., Pau, A., Macdonald, W., Harris, K., Edge, C. J., Franks, N. P., Mahoney, P. F., and Dickinson, R. (2018). Xenon Protects against Blast-Induced Traumatic Brain Injury in an In Vitro Model. J. Neurotrauma 35, 1037-1044.
Campos-Pires, R., Hirnet, T., Valeo, F., Ong, B. E., Radyushkin, K., Aldhoun, J., Saville, J., Edge, C. J., Franks, N. P., Thal, S. C., and Dickinson, R. (2019). Xenon improves long-term cognitive function, reduces neuronal loss and chronic neuroinflammation, and improves survival after traumatic brain injury in mice. Br. J. Anaesth. 123, 60-73.
Campos-Pires, R., Armstrong, S. P., Sebastiani, A., Luh, C., Gruss, M., Radyushkin, K., Hirnet, T., Werner, C., Engelhard, K., Franks, N. P., Thal, S. C., and Dickinson, R. (2015). Xenon improves neurologic outcome and reduces secondary injury following trauma in an in vivo model of traumatic brain injury. Crit. Care Med. 43, 149-158.
Laitio, R., Hynninen, M., Arola, O., Virtanen, S., Parkkola, R., Saunavaara, J., Roine, R. O., Grönlund, J., Ylikoski, E., Wennervirta, J., Bäcklund, M., Silvasti, P., Nukarinen, E., Tiainen, M., Saraste, A., Pietila, M., Airaksinen, J., Valanne, L., Martola, J., Silvennoinen, H., Scheinin, H., Hari ola, V.-P., Niiranen, J., Korpi, K., Varpula, M., Inkinen, O., Olkkola, K. T., Maze, M., Vahlberg, T., and Laitio, T. (2016). Effect of Inhaled Xenon on Cerebral White Matter Damage in Comatose Survivors of Out-of-Hospital Cardiac Arrest: A Randomized Clinical Trial. JAMA 315, 1120-1128.
Jawad, N., Rizvi, M., Gu, J., Adeyi, O., Tao, G., Maze, M., and Ma, D. (2009). Neuroprotection (and lack of neuroprotection) afforded by a series of noble gases in an in vitro model of neuronal injury. Neurosci. Lett. 460, 232-236.
Dingley, J., Tooley, J., Liu, X., Scull-Brown, E., Elstad, M., Chakkarapani, E., Sabir, H., and Thoresen, M. (2014). Xenon ventilation during therapeutic hypothermia in neonatal encephalopathy: a feasibility study. Pediatrics 133, 809-818.
Dworschak, M. (2008). Pharmacologic neuroprotection—is xenon the light at the end of the tunnel? Crit. Care Med. 36, 2477-2479.
Sanders, R. D., Franks, N. P., and Maze, M. (2003). Xenon: no stranger to anaesthesia. Br. J. Anaesth. 91, 709-717.
Shin, S. S., Hwang, M., Diaz-Arrastia, R., and Kilbaugh, T. J. (2021). Inhalational gases for neuroprotection in traumatic brain injury. J. Neurotrauma.
Stoppe, C., Rimek, A., Rossaint, R., Rex, S., Stevanovic, A., Schalte, G., Fahlenkamp, A., Czaplik, M., Bruells, C. S., Daviet, C., and Coburn, M. (2013). Xenon consumption during general surgery: a retrospective observational study. Med. Gas Res. 3, 12.
Law, L. S.-C., Lo, E. A.-G., and Gan, T. J. (2016). Xenon Anesthesia: A Systematic Review and Meta-Analysis of Randomized Controlled Trials. Anesth. Analg. 122, 678-697.
Petersen-Felix, S., Luginbühl, M., Schnider, T. W., Curatolo, M., Arendt-Nielsen, L., and Zbinden, A. M. (1998). Comparison of the analgesic potency of xenon and nitrous oxide in humans evaluated by experimental pain. Br. J. Anaesth. 81, 742-747.
Chattaraj, R., Hammer, D. A., Lee, D., and Sehgal, C. M. (2021). Multivariable Dependence of Acoustic Contrast of Fluorocarbon and Xenon Microbubbles under Flow. Ultrasound Med Biol 47, 2676-2691.
Kilbaugh, T. J., Bhandare, S., Lorom, D. H., Saraswati, M., Robertson, C. L., and Margulies, S. S. (2011). Cyclosporin A preserves mitochondrial function after traumatic brain injury in the immature rat and piglet. J. Neurotrauma 28, 763-774.
Kilbaugh, T. J., Karlsson, M., Byro, M., Bebee, A., Ralston, J., Sullivan, S., Duhaime, A.-C., Hansson, M. J., Elmér, E., and Margulies, S. S. (2015). Mitochondrial bioenergetic alterations after focal traumatic brain injury in the immature brain. Exp. Neurol. 271, 136-144.
Karlsson, M., Pukenas, B., Chawla, S., Ehinger, J. K., Plyler, R., Stolow, M., Gabello, M., Hugerth, M., Elmér, E., Hansson, M. J., Margulies, S., and Kilbaugh, T. (2018). Neuroprotective Effects of Cyclosporine in a Porcine Pre-Clinical Trial of Focal Traumatic Brain Injury. J. Neurotrauma.
Duhaime, A. C., Margulies, S. S., Durham, S. R., O'Rourke, M. M., Golden, J. A., Marwaha, S., and Raghupathi, R. (2000). Maturation-dependent response of the piglet brain to scaled cortical impact. J. Neurosurg. 93, 455-462.
Miao, Y.-F., Peng, T., Moody, M. R., Klegerman, M. E., Aronowski, J., Grotta, J., McPherson, D. D., Kim, H., and Huang, S.-L. (2018). Delivery of xenon-containing echogenic liposomes inhibits early brain injury following subarachnoid hemorrhage. Sci. Rep. 8, 450.
Chattaraj, R., Hwang, M., Zemerov, S. D., Dmochowski, I. J., Hammer, D. A., Lee, D., and Sehgal, C. M. (2020). Ultrasound Responsive Noble Gas Microbubbles for Applications in Image-Guided Gas Delivery. Adv Healthc Mater 9, e1901721.
Borden, M. A., Kruse, D. E., Caskey, C. F., Zhao, S., Dayton, P. A., and Ferrara, K. W. (2005). Influence of lipid shell physicochemical properties on ultrasound-induced microbubble destruction. IEEE Trans Ultrason Ferroelectr Freq Control 52, 1992-2002.
Swanson, E. J., Mohan, V., Kheir, J., and Borden, M. A. (2010). Phospholipid-stabilized microbubble foam for injectable oxygen delivery. Langmuir 26, 15726-15729.
Feshitan, J. A., Legband, N. D., Borden, M. A., and Terry, B. S. (2014). Systemic oxygen delivery by peritoneal perfusion of oxygen microbubbles. Biomaterials 35, 2600-2606.
Shekhar, H., Palaniappan, A., Peng, T., Lafond, M., Moody, M. R., Haworth, K. J., Huang, S., McPherson, D. D., and Holland, C. K. (2019). Characterization and Imaging of Lipid-Shelled Microbubbles for Ultrasound-Triggered Release of Xenon. Neurotherapeutics 16, 878-890.
Borden, M. A., and Song, K.-H. (2018). Reverse engineering the ultrasound contrast agent. Adv Colloid Interface Sci 262, 39-49.
Heurteaux, C., Guy, N., Laigle, C., Blondeau, N., Duprat, F., Mazzuca, M., Lang-Lazdunski, L., Widmann, C., Zanzouri, M., Romey, G., and Lazdunski, M. (2004). TREK-1, a K+ channel involved in neuroprotection and general anesthesia. EMBO J. 23, 2684-2695.
Ma, D., Hossain, M., Pettet, G. K. J., Luo, Y., Lim, T., Akimov, S., Sanders, R. D., Franks, N. P., and Maze, M. (2006). Xenon preconditioning reduces brain damage from neonatal

Additional background can also be found in:
Rajarshi Chattaraj, Daniel A. Hammer, Daeyeon Lee, Chandra M. Sehgal, Multivariable Dependence of Acoustic Contrast of Fluorocarbon and Xenon Microbubbles under Flow, Ultrasound in Medicine & Biology, Volume 47, Issue 9, 2021, Pages 2676-2691;
Misun Hwang, Rajarshi Chattaraj, Anush Sridharan, Samuel S. Shin, Angela N. Viaene, Sophie Haddad, Dmitry Khrichenko, Chandra Sehgal, Daeyeon Lee, and Todd J. Kilbaugh, Neurotrauma Reports. March 2022, 97-104; and
Ultrasound Responsive Noble Gas Microbubbles for Applications in Image-Guided Gas Delivery, Rajarshi Chattaraj,Misun Hwang,Serge D. Zemerov,Ivan J. Dmochowski,Daniel A. Hammer,Daeyeon Lee,Chandra M. Sehgal, Advanced Healthcare Materials (Mar. 24, 2020), https://doi.org/10.1002/adhm.201901721, the entireties of which foregoing references are incorporated herein in their entireties for any and all purposes.

What is claimed:

1. A microbubble composition, comprising:
a plurality of microbubbles,
a microbubble comprising a noble gas and/or perfluorocarbon encapsulated within a shell that comprises one or more of a lipid, a protein, or a polymer, and
a microbubble optionally defining a cross-sectional dimension in the range of from about 0.5 to about 20 micrometers.

2. The microbubble composition of claim 1, wherein the lipid comprises a phospholipid.

3. The microbubble composition of claim 1, wherein the polymer is a lipopolymer.

4. The microbubble composition of claim 1, wherein the shell comprises a phospholipid and a lipopolymer.

5. The microbubble composition of claim 4, wherein the phospholipid comprises 1,2-dipalmitoyl-sn-glycero-3-phosphocholine (DPPC) (C16:0) or 1,2-dibehenoyl-sn-glycero-3-phosphocholine (DSPC) (C22:0).

6. The microbubble composition of claim 3, wherein the lipopolymer comprises polyethylene glycol.

7. The microbubble composition of claim 1, wherein a microbubble defines a cross-sectional dimension in the range of from about 1 to about 10 micrometers.

8. The microbubble composition of claim 1, wherein the noble gas comprises helium, argon, or xenon.

9. The microbubble composition of claim 8, wherein the noble gas comprises xenon.

10. The microbubble composition of claim 8, wherein the shell comprises one or more targeting groups configured to bind to one or more complementary targets.

11. The microbubble composition of claim 10, wherein the targeting group comprises an antibody, a peptide, or an aptamer.

12. The microbubble composition of claim 8, further comprising a carrier fluid in which the plurality of microbubbles is disposed.

13. The microbubble composition of claim 12, wherein the carrier fluid is biocompatible.

14. The microbubble composition of claim 1, wherein the plurality of microbubbles is characterized as being monodisperse.

15. The microbubble composition of claim 1, wherein the plurality of microbubbles is characterized as being polydisperse.

16. A method, comprising: forming a composition according to claim 15.

17. The method of claim 16, wherein the forming comprises agitating the noble gas and/or perfluorocarbon and shell-forming materials.

18. The method of claim 16, wherein the forming comprises applying acoustic energy to an interface between the noble gas and/or perfluorocarbon and shell-forming materials.

19. A method, comprising administering a microbubble composition according to claim 1 to a subject, the composition optionally comprising echogenic phospholipid microbubbles.

20. The method of claim 19, wherein the administration comprises intravenous administration.

21. The method of claim 19, wherein the subject has suffered or is suspected of suffering an injury, the injury optionally being a brain injury.

22. The method of claim 21, further comprising rupturing at least some of the microbubbles.

23. The method of claim 22, the method of claim 22, wherein the rupturing is effected by application of ultrasound energy.

24. The method of claim 22, wherein the microbubbles are ruptured while located in a vessel downstream from the brain.

25. The method of claim 19, further comprising identifying, with application of ultrasound energy, the location of the microbubble composition.

26. A method, comprising: (a) identifying, with the application of energy, the location of a microbubble composition according to claim 1, the energy optionally being ultrasound, (b) controllably effecting rupture of microbubbles of a microbubble composition of claim 1, the rupture optionally being effected by application of ultrasound, or both (a) and (b).

27. The method of claim 26, wherein the microbubbles are disposed within a subject.
